SEQUENCE LISTING (3 sequences)

SEQ ID NO: 1 - protein, 31 aa, unidentified
ACUACAACACUUCACUAAUAAAACAUAAUUA

SEQ ID NO: 2 - protein, 35 aa, unidentified
CUUCACUUCAUUUCCUCCAUCAUACACCUACAUCA

SEQ ID NO: 3 - protein, 52 aa, unidentified
UGAUGUUGUGAAGUGAAGGAGGAUAUAAAUGAAACAGAACAAAGAACAGUUA

(19) United States
(12) Patent Application Publication
(10) Pub. No.: US 2025/0270609 A1
(43) Pub. Date: Aug. 28, 2025

(54) TOEHOLD EXCHANGE RIBOREGULATOR GATE AND REGULATING PROTEIN TRANSLATION WITH TOEHOLD EXCHANGE RIBOREGULATOR

(71) Applicant: Government of the United States of America, as represented by the Secretary of Commerce, Gaithersburg, MD (US)

(72) Inventor: Samuel Wenger Schaffter, Washington, DC (US)

(21) Appl. No.: 19/040,636

(22) Filed: Jan. 29, 2025

Related U.S. Application Data
(63) Continuation-in-part of application No. 18/720,950, filed on Jun. 17, 2024.
(60) Provisional application No. 63/290,457, filed on Dec. 16, 2021.
(60) Provisional application No. 63/626,294, filed on Jan. 29, 2024.

(51) Int. Cl. C12P 21/00; C12N 15/113

(52) U.S. Cl. CPC C12P 21/00; C12N 15/113; C12N 2310/12; C12N 2310/531; C12N 2320/50

(57) ABSTRACT

A toehold exchange riboregulator gate includes: a gate input toehold domain; a ribosome binding sequence; a start codon; a c spacer domain positioned between the ribosome binding sequence and the start codon; a protein coding sequence; and a self-cleaving ribozyme sequence that produces a double-stranded RNA gate suitable for strand exchange and produces the gate input toehold domain.

### CROSS-REFERENCE TO RELATED APPLICATION

This application is a continuation-in-part and claims benefit of the following applications: U.S. patent application Ser. No. 18/720,950 (filed Jun. 17, 2024); and International Patent Application No. PCT/US22/53229 (filed Jan. 1, 2023), both of which claim the benefit of U.S. provisional patent application 63/290,457 (filed Dec. 18, 2022). All patents, patent applications, and publications cited herein are hereby incorporated by reference in their entirety. This application claims the benefit of U.S. Provisional Patent Application Ser. No. 63/626,294 (filed Jan. 29, 2024), which is herein incorporated by reference in its entirety.

### STATEMENT REGARDING FEDERALLY SPONSORED RESEARCH

This invention was made with United States Government support from the National Institute of Standards and Technology (NIST), an agency of the United States Department of Commerce. The Government has certain rights in this invention.

### SEQUENCE LISTING

This application contains a Sequence Listing which has been filed electronically in compliance with ST.26 format and is hereby incorporated by reference in its entirety. The Sequence Listing, created on May 14, 2025, is named 21-053cp1_sequence_listing.xml and is 4 kilobytes in size.

### BACKGROUND

The present invention generally relates to the field of riboregulators, and more particularly to techniques for regulating protein translation with RNA strand exchange reactions with a toehold exchange (THE) riboregulator.

Riboregulators are RNA-based genetic control elements that modulate gene expression. They often involve cis-repressed RNA sequences located in the 5′ untranslated region (UTR) of messenger RNA (mRNA). These sequences can form hairpin structures that sequester the ribosome binding site (RBS), thus preventing ribosome binding and inhibiting translation. Upon interaction with a specific ligand, the RNA structure can change, exposing the RBS and allowing translation to proceed. Various types of riboregulators have been developed, including those responsive to small molecules, temperature changes, light, and nucleic acids. They have found applications in various fields, such as metabolic engineering, diagnostics, and therapeutics. Despite advances in riboregulator design, existing systems have limitations. Many natural riboregulators exhibit limited dynamic range, making it difficult to precisely control protein expression levels. Furthermore, designing synthetic riboregulators that respond to specific ligands or signals can be challenging. Sequence constraints, unpredictable folding behavior, and limited modularity often hinder the development of robust and versatile genetic control elements. These limitations restrict the applicability of riboregulators in complex synthetic biology circuits and sophisticated biotechnological applications. Toehold switches represent an advancement in RNA-based genetic control, offering improved dynamic range and programmability compared to traditional hairpin-based riboregulators. However, toehold switches require increasing complicated structural design to sense multiple inputs or integrate complex logic functions. Moreover, they can exhibit sequence constraints that complicate design, optimization, and protein expression. Addressing these issues is crucial for expanding the utility of RNA-based control systems in synthetic biology and genetic engineering.

It is therefore an objective of the present invention to provide a toehold exchange (THE) riboregulator and process for regulating protein translation with RNA strand exchange reactions with the toehold exchange (THE) riboregulator, thereby overcoming the above-mentioned disadvantages of the prior art at least in part. Accordingly, methods and equipment for using RNA strand exchange reactions for programmable and tunable protein expression would be advantageous and would be favorably received in the art.

### BRIEF DESCRIPTION

One aspect of the present invention relates to a toehold exchange (THE) riboregulator gate. A riboregulator may be understood as an RNA molecule that regulates gene expression. A toehold exchange refers to a strand exchange process initiated by a short single-stranded region called a toehold. It should be appreciated that, more specifically, the exchange refers to both an input toehold and an output toehold. Base pairing with the input toehold initiates strand exchange that subsequently causes the output toehold to unpair, wherein toeholds exchange, such that the input toehold goes from unpaired to pairs and output toehold goes from paired to unpaired.

It may be provided that the toehold exchange (THE) riboregulator gate comprises a gate input toehold domain. A toehold domain is a single-stranded RNA sequence that facilitates strand exchange. A gate input toehold domain initiates the strand exchange reaction, enabling specific recognition of an input RNA molecule. One advantage of this arrangement is its programmability. By designing the sequence of the gate input toehold domain, the riboregulator can be tailored to respond to a wide range of input signals, enhancing its versatility and potential applications in various biological systems. Moreover, this also occurs for an input branch migration domain having a sequence that can be tailored for specificity, wherein even two sequences with the same input toehold do not react unless an output wobble domain matches. With this domain, detection of single nucleotide polymorphisms (SNPs) is practical.

The THE riboregulator gate comprises an output toehold domain. An output toehold domain is a sequence that base pairs with the ribosome binding sequence (RBS) when the gate is in the OFF state. The output toehold domain prevents translation initiation by blocking the ribosome binding sequence. One advantage of this design is the tight control of gene expression. The sequestration of the RBS ensures minimal protein production in the absence of the input strand, thus enhancing the dynamic range of the riboregulator and reducing background noise. The output toehold sequester domain is a sequence genus to the species of RBS and is bound complementary to the output toehold domain. In addition to the output toehold domain sequestering an RBS, the output toehold domain can initiate downstream reactions if released due to strand exchange.

The THE riboregulator gate comprises a ribosome binding sequence. A ribosome binding sequence is an RNA sequence that recruits the ribosome to initiate protein synthesis. One advantage of this is the direct coupling of RNA strand exchange to protein production. This enables a precise and quantifiable readout of riboregulator activity. It also allows the output of RNA computations to be directly linked to cellular functions, opening possibilities for building complex genetic circuits with predictable behavior.

The THE riboregulator gate comprises a start codon. A start codon is the first codon of an mRNA transcript translated by a ribosome. One advantage of this is it defines the translation initiation site. Positioning the start codon downstream of the ribosome binding sequence allows translation to occur only after successful strand exchange. This arrangement ensures precise control of protein production, minimizing leaky expression and improving the fidelity of the genetic control system.

The THE riboregulator gate comprises a c spacer domain. A spacer domain refers to a non-coding RNA sequence that separates functional domains. The c spacer domain is a short sequence located between the output toehold sequester domain (RBS domain) and the start codon. One advantage of this arrangement is optimized translation initiation. The spacer sequence positions the start codon at an ideal distance from the RBS, maximizing translation efficiency and ensuring robust protein production when the riboregulator is activated.

The THE riboregulator gate comprises a protein coding sequence. A protein coding sequence is a series of codons in mRNA that specifies the amino acid sequence of a protein. One advantage of this is it encodes the desired protein product. Coupling the protein coding sequence to the strand exchange mechanism allows any protein of interest to be regulated by the THE riboregulator. This modularity greatly expands the potential applications of the technology, as the protein output can be customized to perform various functions in different cellular contexts.

The THE riboregulator gate comprises a self-cleaving ribozyme sequence that exposes the gate input toehold domain. A self-cleaving ribozyme is an RNA molecule with catalytic activity. It can cleave itself at a specific site. One advantage of this arrangement is efficient production of the functional gate. Co-transcriptional self-cleavage generates a double-stranded RNA gate with an active input toehold domain, which is essential for initiating the strand exchange reaction. It should be appreciated that making the double stranded RNA gate is paramount, and without a gate composed of two RNA strands, strand exchange does not occur, leading to only displacement of a hairpin. This autocatalytic process simplifies the production of the riboregulator, as it eliminates the need for separate processing steps. This streamlines riboregulator synthesis, particularly in cellular environments where complex RNA processing machinery may be slower than desired or may not be readily available.

One aspect of the present invention relates to a process for regulating protein translation with a toehold exchange (THE) riboregulator. Protein translation is the process of creating proteins from an mRNA template. A toehold exchange riboregulator is an RNA molecule that controls gene expression via a strand exchange reaction.

The process comprises contacting the THE riboregulator gate with an input strand. Contacting can include bringing two or more entities into physical proximity. One advantage of this is it initiates the strand exchange reaction. The input strand, by hybridizing to the gate's toehold domain, triggers a cascade of events leading to either the exposure or sequestration of the ribosome binding sequence, thereby modulating protein translation. This interaction provides the trigger for the regulatory mechanism, allowing the system to respond to the presence or absence of the input signal. This bimolecular reaction creates a dynamic system capable of responding to changes in the cellular environment. Further, one can change the lengths of different domains on the input strand to precisely control the protein expression level (translation initiation rate).

### BRIEF DESCRIPTION OF THE DRAWINGS

The following description cannot be considered limiting in any way. Various objectives, features, and advantages of the disclosed subject matter can be more fully appreciated with reference to the following detailed description of the disclosed subject matter when considered in connection with the following drawings, in which like reference numerals identify like elements.

FIG. 1 shows, according to some embodiments, toehold exchange (THE) riboregulators for controlling protein expression.

FIG. 2 shows, according to some embodiments, ctRSD gate and toehold exchange (THE) riboregulator design. Schematic of cotranscriptionally encoded RNA strand displacement (ctRSD) toolkit as operated in vitro. SsRNA inputs and gate hairpins are transcribed from dsDNA templates. An internal self-cleaving ribozyme (Rz) cleaves the gate hairpins to produce dsRNA components that can undergo strand exchange with ssRNA inputs to release ssRNA outputs. The production of outputs is measured with a fluorescent DNA reporter.

FIG. 3 shows, according to some embodiments, ctRSD gate and toehold exchange (THE) riboregulator design. Schematic of a toehold exchange riboregulator which couples RNA strand exchange to protein production. This allows for RNA computation to regulate gene expression and enables measurement of toehold exchange in bacteria. Toehold exchange riboregulators are designed similarly to ctRSD gates except that the output toehold (w:w′) has been changed to a ribosome binding sequence (rbs′:rbs), and the terminator sequence at the 3′ end of the ctRSD gate has been replaced with a protein coding sequence (in this case a fluorescent protein, CFP). After transcription and cleavage of the toehold exchange riboregulator, the ribosome binding sequence is sequestered, preventing translation of the protein coding sequence. If a sequence complementary input is present, strand exchange will displace and output strand from the gate and expose the ribosome binding sequence, initiating translation of the protein coding sequence. This process is referred to as toehold exchange because the base pairing of the input toehold (u) is exchanged for the dissociation of the output toehold (rbs′). THE riboregulators are tested by cloning the input and riboregulator gates on separate plasmids, transforming these plasmids into E. coli, and measuring cellular fluorescence with flow cytometry.

FIG. 4 shows, according to some embodiments, ctRSD gate and toehold exchange (THE) riboregulator design. Sequence schematics of a toehold exchange riboregulator. The s domain is designated as Ns because it can be extended as spacer or, with extensions of the 3′ end of an input, a longer toehold for initiating strand exchange.

FIG. 5 shows, according to some embodiments, characterization of the toehold exchange (THE) riboregulator design space in E. coli. Above the plots, the yellow highlighted domains represent the domains varied in the accompanying panels. MEF indicates molecules of equivalent fluorophore. Results labeled ‘OFF’ represent gates cotranscribed with a non-complementary input. (a,b) Characterizing translation strength of THE riboregulator designs with different d domain lengths for (a) hairpin control constructs that do not require strand exchange or (b) separate input and gate constructs that require strand exchange for gate activation. (c) Bar plots of the geometric means of the cell distributions in (a) and (b). (d,e,f) Characterization of different input toehold lengths (c), input toehold and spacer lengths (d), and output toehold lengths (e). The samples highlighted in yellow on the x-axes indicate the ‘canonical’ designs used in the rest of the study. Inputs and gates were cloned onto separate plasmids with compatible origins of replication (pET and pColA backbones, respectively) and both plasmids were transformed into BL21 Star (DE3) E. coli cells for testing. The input:gate plasmid ratio is expected to be 2:1 based on the origins of replication. Cells were cultured with 100 μM of IPTG to induce T7 RNAP expression for 6 hours prior to flow cytometry. Replicates represent three independent colonies picked after transformation.

FIG. 6 shows, according to some embodiments, toehold exchange riboregulators are sequence specific. (a) Schematics of constructs used to test the sequence specificity of THE riboregulators in E. coli. In sample I., both the toehold and the branch migration domain of the input are not complementary to the gate. In sample II., the input toehold is complementary to gate, but the branch migration domain is not. In samples III. and IV., the input toehold is either missing or not complementary to the gate, respectively. The sample III. results are also shown in panel d of FIG. 5. In sample V., the toehold and branch migration domains of the input are complementary to the gate. (b) Fluorescent cell distributions from flow cytometry and (c) geometric means of fluorescent cell distributions for the constructs depicted in (a). Only when both the toehold and branch migration domains of the input are complementary to the gate is substantial protein expression observed.

FIG. 7 shows, according to some embodiments, ribozyme cleavage is necessary for high protein expression with the canonical toehold exchange riboregulator design. (a) Schematic of the uncleaved ribozyme constructs tested. xR3 has a single mutation relative to R3 that abolishes cleavage activity. The length of the spacer (s) domain was varied within the gate and tested with inputs with either 6 bases (s′=0) or 10 bases (s′=4) of complementarity with the gate toehold. (b) Characterization of uncleaved constructs in (a) with complementary (ON) or non-complementary (OFF) inputs and (c) a comparison of the uncleaved vs cleaved riboregulator gates with complementary inputs. The samples highlighted in yellow on the x-axes indicate the ‘canonical’ designs. When s′=0, there is low protein production for the uncleaved gates. Increasing the spacer length and the complementarity of the input and the gate toeholds increases protein production for the uncleaved gates, likely from loop mediated invasion of the hairpin. Accordingly, ribozyme cleavage is occurring in the canonical THE riboregulator design.

FIG. 8 shows, according to some embodiments, toehold exchange riboregulators can be programmed for different input and output sequences. (a) Schematic of THE riboregulator gates with different input branch migration (BM) domains. The branch migration sequences (1, 3, 4, 5, 6, 7) were taken from the in vitro ctRSD toolkit and directly implemented in THE riboregulators. (b) Fluorescent cell distributions (left) and geometric means from cell distributions (right) for the different branch migration domains shown in (a). Results labeled ‘OFF’ represent gates cotranscribed with a non-complementary input. (c) Characterization of THE riboregulatory gates regulating the expression of different protein coding sequences (CDS). ‘Blank’ indicates cells without any fluorescent protein at the relevant excitation and emission wavelengths. ‘HP’ indicates cells with a constitutively ON hairpin construct shown in FIG. 5A.

FIG. 9 shows, according to some embodiments, orthogonal ribozymes and toeholds from the ctRSD toolkit are functional in THE riboregulators in cells. Schematics of THE riboregulators with different HDV-like ribozymes and spacer sequences and either (a) the u input toehold or (b) the v input toehold. The u and v toeholds and ribozymes sequences were previously characterized in vitro as part of the ctRSD toolkit. The spacer (s) domains were designed with slightly different sequences depending on the ribozyme and input toehold combination to prevent undesired secondary structure predicted by NUPACK. Spacer domains 4u and 4v indicate spacers designed to have additional complementarity with s′ domains on the inputs. All 4s domains are designed with a C base adjacent to the toehold to prevent any pairing with inputs with longer toeholds or outputs from upstream gates that serve as inputs. (c) Geometric means of fluorescent E. coli distributions from flow cytometry for the constructs shown in panels a and b.

FIG. 10 shows, according to some embodiments, ctRSD circuits operate across environments. Here, ctRSD circuits operate across environments. The bottom panel shows experimental data of ctRSD circuits operating in (from left to right) simple in vitro transcription reactions, PURExpress cell-free transcription-translation reactions, BL21 Star (DE3) bacterial cell lysate, and BL21 Star (DE3) cells.

FIG. 11 shows, according to some embodiments, toehold exchange (THE) riboregulators for tunable protein expression.

FIG. 12 shows, according to some embodiments, toehold exchange (THE) riboregulators in E. coli cells.

FIG. 13 shows, according to some embodiments, toehold exchange (THE) riboregulators involving varying length and complementarity of domains.

FIG. 14 shows, according to some embodiments, toehold exchange (THE) riboregulators involving varying output protein sequences.

FIG. 15 shows, according to some embodiments, toehold exchange (THE) riboregulators involving multilayer cascades.

FIG. 16 shows results demonstrating the use of a toehold exchange riboregulator to control cell growth rate (fitness) across different antibiotic (trimethoprin, TMP) concentrations. Io is a scrambled input that cannot initiate strand exchange. DHFR is an essential gene for growth and inhibited by the antibiotic TMP. By regulating a copy of E. coli DHFR on a toehold exchange riboregulator, we can control the levels of TMP the cells. Data points represent three biological replicates conducted on different different days. Solid lines represent the mean and shaded regions represent one standard deviation of the mean. Growth rates at each TMP concentration were normalized to the growth rate of the same strain without TMP. BL21* (DE3) cells were grown overnight in LB media with antibiotic, diluted 50-fold and grown for 4 hrs with 100 μmol/L of IPTG at 37° C. prior to TMP addition to the media.

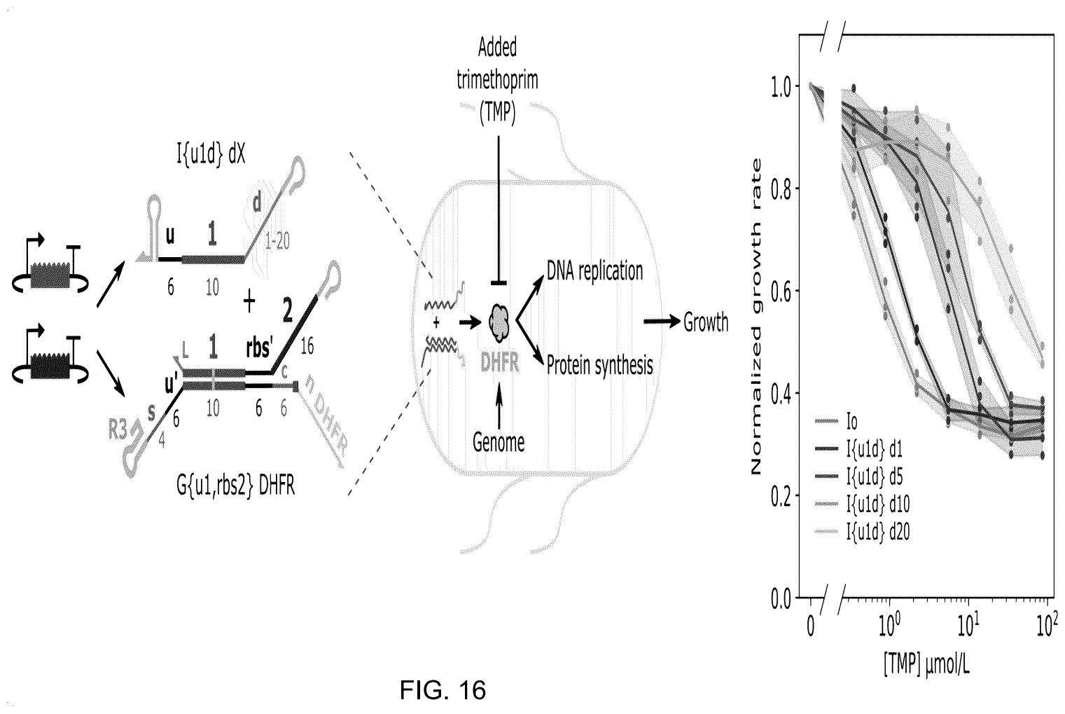

FIG. 17 shows results demonstrating a two-layer cascade of a ctRSD gate reacting with an input strand to produce an output strand that in turn reacts with a toehold exchange riboregulator to produce CFP signal. Experiments were conducted with three different input sequences (domain 3, 4, or 5) as well as with ctRSD gates (G{vX,u1d}) that possess a linker sequence (L) that is single stranded (-hp) or a hairpin structure (hp4). The gates with hp4 produced higher CFP signal across all input sequences. Experiments conducted in BL21* (DE3) E. coli as described in previous figures. Gate strand and input strand were encoded sequentially on the same plasmid.

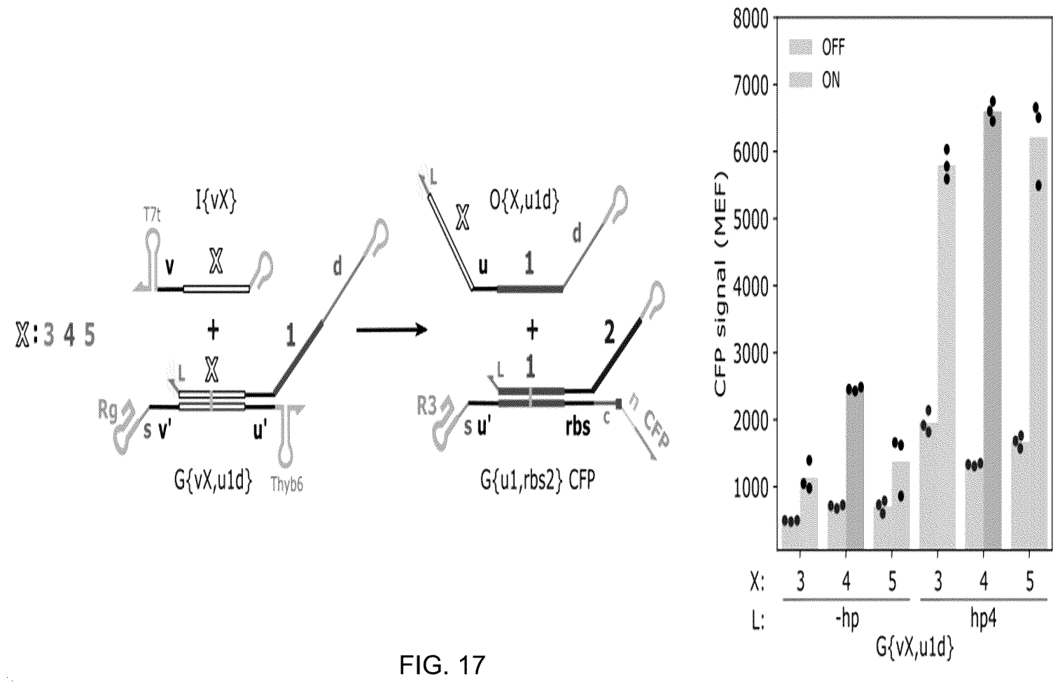

### DETAILED DESCRIPTION

A detailed description of one or more embodiments is presented herein by way of exemplification and not limitation.

Conventional riboregulators, particularly those based on hairpin structures, often suffer from limited dynamic range and can be challenging to design for specific applications. Their reliance on unimolecular conformational changes restricts their modularity and potential for complex logic functions. Traditional toehold switches, while offering improvements in dynamic range, still face limitations in sensing multiple inputs and integrating complex logic. Further, sequence constraints and reliance on single-stranded RNA inputs can pose design and delivery challenges.

The toehold exchange (THE) riboregulator gate described herein overcomes these limitations by employing a bimolecular strand exchange mechanism, offering precise control over protein expression and enhanced modularity for sensing diverse inputs. It has been discovered that a toehold exchange (THE) riboregulator gate offers significant advantages. One advantage is its ability to precisely modulate protein expression strength. The gate input toehold domain and output toehold sequester domain work together to regulate translation initiation. The gate input toehold domain initiates the strand exchange reaction upon interaction with a specific input RNA molecule, leading to the displacement of the output strand and exposure of the ribosome binding sequence. This bimolecular reaction provides a higher degree of control compared to traditional hairpin-based riboregulators, enabling fine-tuning of protein expression levels. Further, the inclusion of the self-cleaving ribozyme sequence enhances the efficiency of riboregulator production, particularly in cellular environments. Accordingly, the self-cleaving ribozyme sequence provides the ability to make the dsRNA gate for toehold exchange. Without the self-cleaving ribozyme, strand exchange may not occur. Using the self-cleaving ribozyme is one of the most efficient ways to do this because the ability to produce the gate is entirely encoded in its sequence and does not involve additional cellular machinery. Other ways mechanisms to produce the dsRNA gate are contemplated and should be appreciated by one skilled in the art.

Co-transcriptional self-cleavage generates the gate input toehold domain, simplifying the synthesis process and eliminating the need for separate processing steps. This streamlined production improves the overall robustness and scalability of the system. Moreover, the modular design of the THE riboregulator, with distinct input, gate, and output domains, facilitates its integration into complex genetic circuits and expands its applicability in various synthetic biology applications. The predictable kinetics of toehold exchange reactions, combined with the ability to rationally design sequences for specific inputs and protein outputs, offers unprecedented control over cellular behavior. The toehold exchange mechanism provides a versatile platform for building sophisticated genetic control systems with improved performance and modularity.

In an embodiment, a toehold exchange (THE) riboregulator gate (225) comprises: a gate input toehold domain (210); an output toehold sequester domain (213) comprising a ribosome binding sequence (226); a start codon (218); a c spacer domain (217) positioned between domain (213) and the start codon (218); a protein coding sequence (220); and a self-cleaving ribozyme sequence (209) that produces domain (210). In an embodiment, the THE riboregulator gate (225) further comprises: an output strand (201) comprising: an input branch migration domain (206); an output branch migration domain (204); an output toehold domain (205) positioned between domain (206) and domain (204); a hairpin-forming sequence (203) connected to domain (204); an output wobble domain (207) connected to domain (206); and a linker (L) sequence (208) connected to domain (206); and a gate prime strand (202) hybridized to the output strand (201) comprising: a gate prime wobble domain (212) connected to domain (211); domain (213) connected to domain (209); a substrate domain (211) connected to domain (210), and a transcription termination sequence (214) connected to domain (213). In an embodiment, the gate prime wobble domain (212) is connected to the substrate domain (211) through guanine-uracil (G-U) wobble base pairings. In an embodiment, a portion of the input branch migration domain (206) is connected to the output wobble domain (207) through G-U wobble base pairings. In an embodiment, the output strand (201) further comprises a toehold spacer domain (221). In an embodiment, the self-cleaving ribozyme sequence (209) is a hepatitis delta virus (HDV) ribozyme. In an embodiment, the output toehold sequester domain (213) is a bacterial ribosome binding sequence. In an embodiment, the start codon (218) is AUG, GUG, or UUG. In an embodiment, the c spacer domain (217) is 5 to 7 bases in length. In an embodiment, the protein coding sequence (220) encodes for a fluorescent protein, an enzyme, or a growth factor. In an embodiment, the self-cleaving ribozyme sequence (209) is any ribozyme that effectuates self-cleaving, e.g., a ribozyme with an HDV-like fold.

The gate input toehold domain (210) is a single-stranded RNA sequence that initiates the strand exchange reaction. It interacts with the input strand (215), triggering the displacement of the output strand (201) and subsequent exposure of the ribosome binding sequence (226). This domain can be implemented using any RNA sequence that is complementary to the input toehold domain (223) of the input strand. One benefit of this domain is its programmability. Different sequences can be used to recognize specific input strands, enabling the riboregulator to respond to diverse signals. Variations in toehold length and sequence can fine-tune the kinetics of strand exchange, optimizing the system's response time and sensitivity.

The output toehold sequester domain (213) is a ribosome binding sequence (226) which is sequestered by the output toehold (205) when the gate is in the OFF state (225). This prevents translation initiation by blocking ribosome access. This domain can be implemented using any RNA sequence that is complementary to the RBS. The key benefit of this domain is its tight control over protein translation. The sequestration of the RBS ensures minimal protein production in the absence of the input strand, maximizing the dynamic range of the riboregulator. Alternative implementations could involve different sequestering mechanisms, such as RNA aptamers or protein-binding sequences, that block translation initiation through steric hindrance or recruitment of inhibitory factors.

The ribosome binding sequence (226) recruits the ribosome to initiate protein synthesis. Its accessibility determines whether translation proceeds. This sequence is typically a short, conserved sequence found in bacterial mRNAs. One benefit of this sequence is the direct coupling of RNA strand exchange to protein production. This allows for precise control of protein levels and provides a readily measurable output. Variations in the RBS sequence can affect translation efficiency, offering an additional layer of control over protein expression.

The start codon (218), typically AUG, marks the beginning of the protein coding sequence. Its position downstream of the output toehold sequester domain ensures translation only occurs after successful strand exchange. This codon is essential for initiating protein synthesis. Its precise placement relative to the RBS (226) ensures tight regulation of translation, minimizing leaky expression and maximizing the dynamic range of the riboregulator.

The c spacer domain (217) is a short sequence between the RBS (226) and the start codon (218). It optimizes the spacing between these elements for efficient translation initiation. This spacer can be implemented using any RNA sequence that does not interfere with ribosome binding or translation initiation. One benefit of this spacer is enhanced translation efficiency. The length and sequence of the spacer can be optimized to fine-tune translation rates, allowing for precise control over protein production.

The protein coding sequence (220) determines the amino acid sequence of the translated protein. It is placed downstream of the start codon and can encode any protein of interest. The benefit of this sequence is modularity. The riboregulator can be designed to control the expression of any protein by simply changing the protein coding sequence. This versatility makes the THE riboregulator applicable in diverse biological systems and for various genetic engineering applications.

The self-cleaving ribozyme sequence (209) is an RNA sequence with catalytic activity that produces the gate input toehold domain (210). It autocatalytically cleaves itself, generating the gate input toehold domain during transcription. Here, the input toehold domain is produced, and the ribozyme cleaving activates the toehold or gate for strand exchange by producing a dsRNA complex. This ribozyme can be implemented using various self-cleaving RNA motifs, such as the HDV ribozyme. One benefit is streamlined production of the functional gate. Co-transcriptional cleavage simplifies the synthesis process, eliminating the need for separate enzymatic processing steps. This is particularly advantageous in cellular environments, improving the efficiency and robustness of THE riboregulator production.

The specific implementation of each element in the THE riboregulator gate contributes to its improved performance and versatility. The gate input toehold domain's programmability enables tailored responses to diverse input signals. The output toehold domain and ribosome binding sequence provide tight control of gene expression, maximizing dynamic range. It should be appreciated that the output toehold domain (205) sequesters the RBS (226). The start codon and c spacer domain ensure precise translation initiation, while the protein coding sequence allows modular control of any protein of interest. Finally, the self-cleaving ribozyme streamlines riboregulator production, improving efficiency and scalability. These combined advantages establish the THE riboregulator gate as a powerful and versatile tool for genetic control in synthetic biology.

The output strand (201) comprises functional domains essential for the strand exchange reaction. The input branch migration domain (206) hybridizes to the substrate domain (211) of the gate prime strand (202), holding the dsRNA THE gate together. The output branch migration domain (204) allowing for downstream interactions. The output toehold domain (205), input strand is sequestered by the output toehold sequester domain (213) preventing it from reacting downstream. The output toehold sequester domain (213) can be designed specifically to be an RBS (226), in which case it is sequestered by the output toehold domain (205) to prevent ribosome initiation. The hairpin-forming sequence (203) stabilizes the output strand and prevents premature degradation. The output wobble domain (207) and linker (L) sequence (208) can be designed to optimize folding and stability. These domains can be implemented using RNA sequences of varying lengths and nucleotide compositions. Variations could include alternative secondary structures or the addition of other functional domains, such as aptamers or protein-binding sites.

The gate prime strand (202) contains sequences that interact with both the input strand (215) and the output strand (201). The gate prime wobble domain (212) interacts with the substrate domain (211), influencing the kinetics of strand exchange. The output toehold sequester domain (213) base pairs with the output toehold domain (205) to prevent the output strand from reacting downstream. If the output toehold sequester domain is designed to be a ribosome binding sequence (226) then it is sequestered by the output toehold domain (205) in the OFF state (225), preventing translation. The substrate domain (211), connected to the gate input toehold domain (210), provides a complementary region for the input strand. The transcription termination sequence (214) signals the end of transcription. These domains can be implemented using various RNA sequences, typically composed of an RNA hairpin followed by a string of U bases.

In some embodiments, the gate prime wobble domain (212) can be connected to the substrate domain (211) through G-U wobble base pairings. These non-canonical base pairs provide flexibility in the RNA structure, influencing the kinetics of strand exchange and optimizing the riboregulator's response to the input signal.

The input branch migration domain (206) can be connected to the output wobble domain (207) through G-U wobble base pairs, further modulating RNA structure and stability. This configuration can influence the efficiency of the strand exchange reaction.

The output strand (201) may include a toehold spacer domain (221), offering additional flexibility in optimizing the interactions between the riboregulator components. This domain can be of variable length and sequence, allowing fine-tuning of the system's response.

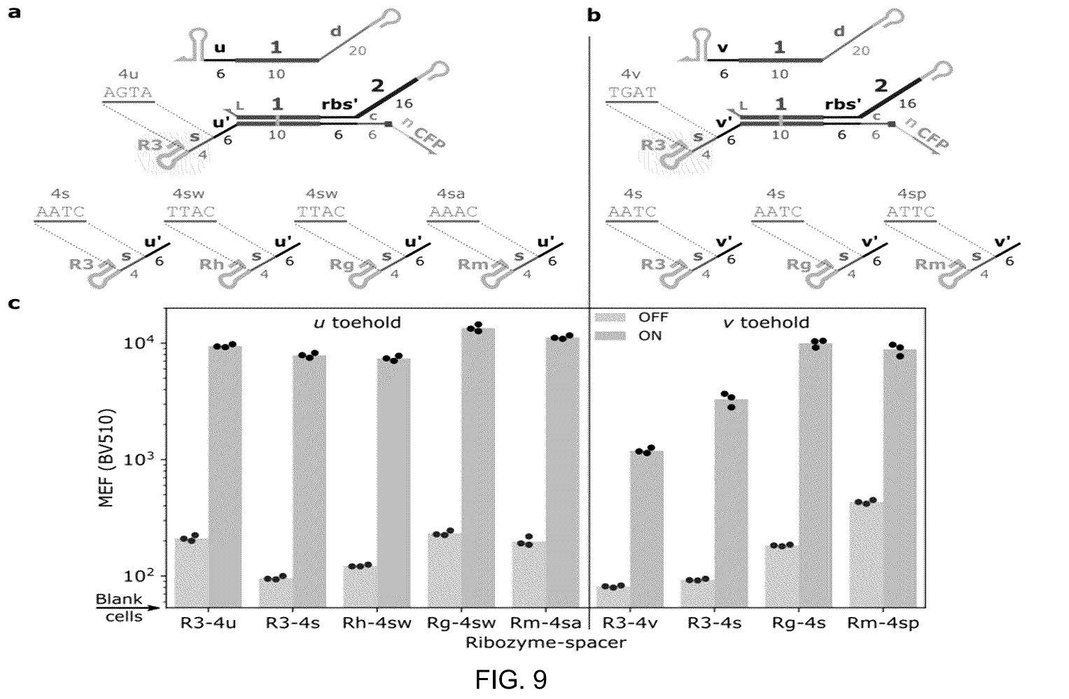

The self-cleaving ribozyme sequence (209) can be a hepatitis delta virus (HDV) ribozyme, known for its efficient self-cleavage activity, or other ribozymes with HDV-like folds, as shown in FIG. 9. This implementation ensures robust production of the gate input toehold domain.

The output toehold domain (205) can be designed to be complementary to a bacterial ribosome binding sequence. This specific implementation enhances the sequestration of the RBS, ensuring tight control of translation initiation.

The start codon (218) can be implemented using the standard codons AUG, GUG, or UUG, providing compatibility with various biological translation systems.

A c spacer domain (217) of 5 to 7 bases provides optimal spacing between the ribosome binding sequence 226 and the start codon, maximizing translation efficiency.

The protein coding sequence (220) can encode various proteins, including fluorescent proteins, enzymes, and growth factors, showcasing the modularity of the THE riboregulator.

The incorporation of these specific elements in the THE riboregulator gate and process offers numerous technical advantages. The G-U wobble base pairings provide flexibility and control over RNA structure and kinetics. The use of an HDV ribozyme ensures efficient self-cleavage and gate production. Complementarity to a bacterial ribosome binding sequence enhances RBS sequestration and regulatory control. The variable spacer domain length, standard start codons, and modular protein coding sequence offer flexibility and adaptability to different applications. These features enhance the performance, versatility, and controllability of the THE riboregulator system, making it a valuable tool in synthetic biology and genetic engineering.

It should be appreciated that strand exchange is distinct from strand displacement used in other technologies such as toehold switches. Herein, exchange refers to exchange of which strand is bound to the gate. In the OFF state, the output strand is bound to the gate. In the ON state, the input strand is bound, and the output strand is not bound. Input binding is exchanged for output release.

The co-transcriptional RNA strand displacement (ctRSD) gate (200) is an RNA-based molecular device that functions as a logic gate. It is designed to control the release of an output RNA strand (201) in response to the presence of a specific input RNA strand (215). The ctRSD gate (200) includes two RNA strands: the output strand (201) and the gate prime strand (202). These strands are partially complementary and hybridize to form a double-stranded RNA structure. The gate prime strand (202) contains a self-cleaving ribozyme sequence (209) that generates a gate input toehold domain (210) upon cleavage. This toehold domain (210) is essential for initiating the strand exchange reaction with the input strand (215). The ctRSD gate (200) operates by undergoing a strand exchange reaction. When the input strand (215) is present, it binds to the gate input toehold domain (210), initiating branch migration and displacing the output strand (201). The released output strand can then participate in downstream reactions, such as activating a reporter gene or triggering another ctRSD gate (200). One benefit of the ctRSD gate is its ability to be produced cotranscriptionally, such that the gate self-assembles during RNA transcription. This simplifies the manufacturing process and allows for genetic encoding of the gate. Variations in sequence length and design can be used to tune the kinetics of strand exchange. The ctRSD gate can be used to build complex logic circuits and integrate them with cellular processes. The ctRSD gate (200), through its co-transcriptional self-assembly and strand exchange mechanism, provides a versatile platform for controlling RNA-based reactions. Its ability to be genetically encoded and integrated with cellular processes opens possibilities for building sophisticated synthetic biology circuits with predictable and programmable behavior. The co-transcriptional RNA strand displacement (ctRSD) gate (200) is also described in U.S. patent application Ser. No. 18/720,950, the disclosure of which is incorporated herein in its entirety.

The output strand (201) is one of the two RNA strands that form the ctRSD gate (200). It contains several functional domains that play critical roles in the strand exchange reaction. These include the input branch migration domain (206), output branch migration domain (204), output toehold domain (205), hairpin-forming sequence (203), output wobble domain (207), and linker (L) sequence (208). The input and output branch migration domains (206, 204) are partially complementary to the input strand (215) and are involved in the strand exchange process. The output toehold domain (205) provides an initial binding site for the input strand, promoting efficient strand exchange. The hairpin-forming sequence (203) stabilizes the output strand's structure, while the output wobble domain (207) and linker sequence (208) can be designed to optimize folding and stability. One benefit of the output strand's modular design is it allows for customization of the strand exchange reaction. The lengths and sequences of the different domains can be tailored to tune the kinetics and thermodynamics of the reaction, achieving specific control over output release. Alternative implementations could include incorporating aptamers or protein-binding sites for additional functionality. The modular design of the output strand (201), with its distinct functional domains, allows for precise control over the strand exchange reaction and facilitates customization for specific applications. The output strand's structural features, including the hairpin-forming sequence and wobble domain, enhance its stability and functionality, contributing to the robustness of the ctRSD gate.

The gate prime strand (202) is the second RNA strand that forms the ctRSD gate (200), hybridizing with the output strand (201) to form the gate structure. It contains the self-cleaving ribozyme sequence (209), gate input toehold domain (210), substrate domain (211), gate prime wobble domain (212), output toehold sequester domain (213) or ribosome binding sequence (226), and transcription termination sequence (214). The self-cleaving ribozyme (209) generates the gate input toehold domain (210) during transcription, initiating gate formation. The substrate domain (211) is complementary to the input branch migration domain of the output strand, while the ribosome binding sequence (226) base pairs with the output toehold (205) of the output strand, initially preventing translation. The gate prime wobble domain (212) modulates the kinetics of strand exchange. One benefit of this strand is its integration of multiple functions. It combines gate formation, input recognition, output sequestration, and translational control into a single molecule. This multi-functionality streamlines the regulatory process. Alternative implementations could involve different ribozymes or sequestering mechanisms. The gate prime strand (202) plays a crucial role in the functionality of the ctRSD gate by combining several key functions into a single molecule. The self-cleaving ribozyme ensures efficient gate formation, while the toehold, substrate, and sequester domains contribute to controlled strand exchange and regulation of translation.

The hairpin-forming sequence (203) is located at the 3′ end of the output strand (201). This sequence folds back on itself to form a hairpin structure, which stabilizes the output strand and can prevent premature degradation. The sequence can be designed with varying stem lengths and loop sizes to optimize stability. The primary benefit of this sequence is enhanced stability of the output strand. The hairpin structure protects the output strand from degradation by cellular RNases, increasing its lifespan and ensuring its availability for downstream reactions. Alternative implementations could include other stabilizing structures, such as G-quadruplexes or protein-binding motifs. The hairpin-forming sequence (203), through its ability to form a stable hairpin structure, provides crucial protection to the output strand against degradation, increasing its lifespan and ensuring its availability for downstream reactions. This enhanced stability contributes to the overall robustness and reliability of the ctRSD gate.

The output branch migration domain (204) is a sequence on the output strand (201) that connects the output strand of a gate to downstream components, such as other gates. One benefit of this domain is that it allows multiple gates to be integrated together to perform information processing and logic.

The output toehold domain (205) is a short, single-stranded RNA sequence located between the input and output branch migration domains (206, 204) on the output strand (201). For the THE riboregulator, this domain blocks the RBS and prevents translation. It controls whether the output strand (201) can react with other downstream components. If the output toehold domain is sequestered then the output strand of the gate cannot react downstream, if the output toehold domain is released due to strand exchange, the output strand can react downstream. This domain is typically 4 to 12 nucleotides long and its sequence is designed to be complementary to a region of the gate prime strand known as the output toehold sequester domain (213), which in some embodiments can be a ribosome binding sequence (226). One benefit of the output toehold domain (205) is its role in regulating the rate and specificity of an RNA strand exchange reaction, ensuring a rapid and controlled response to the input signal. The length and sequence of the toehold can be modified to adjust the kinetics of strand exchange, allowing for fine-tuning of the system's sensitivity and response time. This toehold-mediated initiation enhances the rate and specificity of the reaction, ensuring a rapid and controlled response to the presence of the input signal. It should be appreciated that the output toehold domain 205 is a sequence that is a genus to the ribosome binding sequence (RBS) sequestering domain 228, and the output toehold domain 205 is complementary to the output toehold sequester domain 213. That is, the output toehold domain 205 is the general term for this domain on ctRSD gates, and the ribosome binding sequence (RBS) sequestering domain 228 is a specific type of sequence for use in a THE riboregulator.

The input branch migration domain (206) is a sequence on the output strand (201) that is partially complementary to the substrate domain (211) on the gate prime strand (202). This domain plays a critical role in the strand exchange process. When the input strand (215) binds to the input toehold domain (210), it initiates branch migration between the input branch migration domain (206) and the substrate domain (211). This branch migration process facilitates the displacement of the output strand (201) from the gate prime strand (202). The length and sequence of the input branch migration domain (206) can be varied to adjust the kinetics and specificity of strand exchange. One benefit of this domain is its contribution to the controlled release of the output strand. The degree of complementarity between the input branch migration domain and the substrate domain can be fine-tuned to achieve specific rates of strand exchange. Variations can include the introduction of mismatches or wobble base pairs to modulate the stability of the duplex. The input branch migration domain (206) plays an essential role in regulating the strand exchange process, facilitating the controlled release of the output strand in response to the input signal. Its interaction with the substrate domain enables precise tuning of the reaction kinetics, optimizing the system's performance.

The output wobble domain (207) is a specialized sequence within the input branch migration domain (206) of the output strand (201). It contains one or more G-U wobble base pairings, which are non-canonical base pairs that can occur in RNA. These wobble base pairs (207) introduce thermodynamic instability into the RNA duplex, promoting strand exchange and facilitating the release of the output strand (201). A benefit of incorporating wobble base pairs is it facilitates the strand exchange process by reducing the energy required to separate the output strand from the gate prime strand. This contributes to the overall efficiency and sensitivity of the THE riboregulator system. The number and position of G-U wobble base pairings can be optimized to achieve desired strand exchange kinetics. Alternatives to G-U wobbles could include other non-canonical base pairs or mismatches. The output wobble domain (207), by introducing thermodynamic instability through G-U wobble base pairings, enhances the efficiency of strand exchange and facilitates the release of the output strand. This contributes to the sensitivity and responsiveness of the THE riboregulator system.

The linker (L) sequence (208) is a non-coding RNA sequence that connects the output strand (201) to the self-cleaving ribozyme (209). This linker (208) provides structural and sequence flexibility to facilitate proper ribozyme folding and self-cleavage. The length and sequence of the linker can generally be varied without affecting its functionality, as long as it does not interfere with the folding or interaction of other domains. One benefit of the linker sequence (208) is that it can be designed to be different RNA stabilizing hairpin sequences to prevent degradation of the output strand in cellular environments. The linker (L) sequence (208) plays an essential structural role, providing flexibility for optimal ribozyme activity. This contributes to the overall efficiency and adaptability of the THE riboregulator.

The self-cleaving ribozyme sequence (209) is a catalytic RNA sequence located within the gate prime strand (202). It cleaves itself at a specific site, generating the double stranded RNA gate (200,225) during transcription. This autocatalytic process eliminates the need for separate enzymatic processing steps, simplifying THE riboregulator production. The HDV ribozyme is a common choice for its efficient self-cleavage activity. One benefit of using a self-cleaving ribozyme is the streamlining of gate production. Co-transcriptional cleavage ensures the efficient generation of the double stranded RNA gate that is essential for the strand exchange reaction. This simplifies the manufacturing process and improves overall efficiency, particularly in cellular environments. Alternative ribozymes, such as the Twister or Pistol ribozyme, could also be employed, offering flexibility in design and optimization. The self-cleaving ribozyme sequence (209), through its autocatalytic activity, streamlines the production of the THE riboregulator gate, enhancing efficiency and enabling genetic encoding of the system. This self-processing feature is particularly advantageous in cellular contexts where complex RNA processing machinery may not be readily available.

The gate input toehold domain (210) is a single-stranded RNA sequence generated by the self-cleavage of the ribozyme sequence (209). It serves as the binding site for the input toehold domain (223) on the input strand (215). This interaction initiates the strand exchange reaction. This domain (210) can be implemented using RNA sequences complementary to the input toehold domain. The length and sequence of the toehold (210) can be modified to tune the kinetics of strand exchange, optimizing the system's response. One benefit of this domain is precise input recognition. The sequence of the gate input toehold domain determines the specificity of the strand exchange reaction. It ensures that only the intended input strand can trigger the regulatory process, minimizing off-target effects and enhancing the fidelity of the system. Variations in toehold length can be used to modulate the sensitivity and kinetics of input recognition. The gate input toehold domain (210), through its specific interaction with the input strand, ensures precise input recognition and triggers the strand exchange reaction, playing a crucial role in the initiation of the regulatory process. Its programmable nature allows for customization of the riboregulator to respond to diverse input signals.

The substrate domain (211) is a sequence on the gate prime strand (202) that is partially complementary to the input branch migration domain (206) on the output strand (201). This domain is involved in the strand exchange process. Following toehold binding, the input branch migration domain (206) of the input strand (215) hybridizes to the substrate domain (211), initiating branch migration and displacement of the output strand. The length and sequence of the substrate domain (211) can be designed to optimize the kinetics of strand exchange, achieving controlled release of the output strand. One benefit of this domain is controlled output release. The extent of complementarity between the substrate domain (211) and the input branch migration domain (206) influences the rate of strand exchange. This feature allows for fine-tuning of the timing and magnitude of output release, providing precise control over the regulatory process. Variations can include incorporating mismatches or non-canonical base pairs to modulate the stability of the duplex and the rate of branch migration. The substrate domain (211), through its controlled interaction with the input branch migration domain (206), plays an essential role in regulating the rate and efficiency of strand exchange. This precise control over output release is crucial for achieving specific regulatory outcomes and fine-tuning the system's response.

The gate prime wobble domain (212) is a sequence on the gate prime strand (202) that is partially complementary to the substrate domain (211). Similar to the output wobble domain (207), it may contain one or more G-U wobble base pairings, introducing thermodynamic instability into the RNA duplex. This instability promotes strand exchange and facilitates the release of the output strand (201). The number and position of G-U wobble base pairs can be modified to fine-tune the kinetics of the reaction. One benefit of this domain is enhanced strand exchange. The wobble base pairs reduce the energy required to separate the output strand from the gate prime strand, increasing the efficiency of the strand exchange reaction and improving the sensitivity of the THE riboregulator. Alternative implementations could include other non-canonical base pairs or mismatches. The gate prime wobble domain (212), through the introduction of G-U wobble base pairings, reduces the thermodynamic stability of the RNA duplex, enhancing strand exchange and facilitating output release. This contributes to the sensitivity and responsiveness of the THE riboregulator system.

The output toehold sequester domain (213) is an RNA sequence on the gate prime strand (202) that is complementary to the output toehold domain (205) of the output strand (201). This domain sequesters the output toehold so that the output strand will not react with other components in the absence of strand exchange. One benefit of this domain is tight control over how the output strand of the gate interacts with other components. It should be appreciated that this domain is, more specifically, a ribosome binding sequence 226 to regulate protein expression. It should be appreciated that the output toehold sequester domain 213 is a sequence that is a genus to that ribosome binding sequence 226, and the output toehold sequester domain 213 is complementary to output toehold domain 205. That is, the output toehold sequester domain 213 is the general term for this domain on ctRSD gates, and the ribosome binding sequence 226 is a specific type of sequence for use in a THE riboregulator.

The transcription termination sequence (214) is located at the 3′ end of the gate prime strand (202). This sequence signals the end of RNA transcription, ensuring that the gate prime strand is of the correct length and contains all the necessary functional domains. Various termination sequences can be used, depending on the RNA polymerase and expression system employed. One benefit of a defined termination sequence is it ensures accurate production of the gate prime strand. Precise termination is crucial for generating a functional riboregulator. Premature termination could result in a truncated gate prime strand lacking essential domains. Variations in termination sequence strength and efficiency can influence transcription termination. The transcription termination sequence (214) plays a crucial role in controlling the length and accuracy of the transcribed gate prime strand. This ensures the inclusion of all necessary functional domains, contributing to the production of a functional THE riboregulator gate.

The specific implementation of each element in the THE riboregulator gate contributes to its improved performance and versatility. The output strand (201) with its multiple domains enables a controlled strand exchange reaction and connection to other components or gates. The gate prime strand (202) integrates gate formation, input recognition, and output sequestration. The hairpin-forming sequence (203) enhances output strand stability, while the wobble domains (207, 212) and linker sequence (208) optimize folding and interactions. A precisely defined RBS (226) ensures efficient translation initiation, while the output toehold domain (205) provides tight control over translation. The self-cleaving ribozyme (209) streamlines gate production, and the transcription termination sequence (214) ensures accurate RNA synthesis. These combined features enhance the functionality, controllability, and applicability of the THE riboregulator system.

The input strand (215) is a single-stranded RNA molecule that serves as the trigger for the THE riboregulator (225). It contains the input toehold domain (223), input branch migration domain (206), and distal (d) ribosome standby domain (222). The input toehold domain (223) initiates strand exchange by binding to the gate input toehold domain (210) on the gate prime strand (202). The input branch migration domain (206) then hybridizes to the substrate domain (211) on the gate prime strand, facilitating strand exchange. The distal ribosome standby domain (222) can modulate the kinetics of protein translation from the THE riboregulator in the ON state. This domain can be of variable length, typically from 0 to 20 nucleotides, with longer sequences increasing the rate of protein translation, and its sequence can be customized without generally influencing system performance. One benefit of the input strand (215) is its programmable nature. The sequence of the input toehold domain determines the specificity of the riboregulator's response, enabling targeted activation by particular RNA signals. Variations in the length of the distal ribosome standby domain allow for fine-tuning of translation kinetics and optimization of the riboregulator's performance. The input strand (215), with its distinct functional domains, provides a specific and programmable trigger for the THE riboregulator. The input toehold domain initiates the strand exchange reaction, while the distal ribosome standby domain allows for fine-tuning of protein production rate. This programmability and controllability make the input strand a versatile tool for regulating gene expression.

The strand exchange product (216) is the RNA complex formed after the strand exchange reaction between the THE riboregulator gate (225) and the input strand (215). It consists of the input strand (215) hybridized to the gate prime strand (202), having displaced the output strand (201). This new RNA complex represents the ON state (227) of the riboregulator, as the ribosome binding sequence (226) is now exposed, allowing translation initiation. One benefit of forming the strand exchange product (216) is activation of the THE riboregulator. The displacement of the output strand exposes the RBS, triggering protein translation. This transition from the OFF state (225) to the ON state (227) enables precise control over protein production in response to the input signal. The stability of the strand exchange product can be influenced by the design of the toehold and branch migration domains, allowing for customization of the riboregulator's response. The formation of the strand exchange product (216) marks the transition of the THE riboregulator from the OFF state to the ON state, enabling protein translation. This transition is critical for the regulation of gene expression in response to the input signal. The stability and structure of this product contribute to the robustness and controllability of the system.

The c spacer domain (217) is a short, non-coding RNA sequence located between the output toehold sequester domain (213) and the start codon (218) on the gate prime strand (202). This spacer sequence (217) plays a critical role in optimizing translation initiation. It ensures the correct spacing between the ribosome binding sequence (226) and the start codon, maximizing translation efficiency when the riboregulator is in the ON state (227). The length of the c spacer domain (217) is typically between 5 and 7 nucleotides. One benefit of the c spacer domain is enhanced translation efficiency. By precisely positioning the start codon relative to the RBS, the spacer sequence promotes efficient ribosome binding and translation initiation, maximizing protein production. Variations in spacer length and sequence can fine-tune translation rates, optimizing the system's performance. The c spacer domain (217), through its optimized length and positioning, enhances translation initiation efficiency, maximizing protein production when the THE riboregulator is activated. This precise spacing contributes to the overall performance and robustness of the system.

The start codon (218), typically AUG, is a three-nucleotide sequence on the gate prime strand (202) that signals the beginning of the protein coding sequence (220). It is located immediately downstream of the c spacer domain (217). This codon (218) plays a critical role in initiating protein translation. When the ribosome binding sequence (226) is exposed after strand exchange, the ribosome binds to the RBS and initiates translation at the start codon. One benefit of the start codon is controlled protein production. Its placement downstream of the output toehold domain (205), which sequesters the RBS, ensures that translation only occurs when the riboregulator is in the ON state (227), preventing leaky expression and maximizing the dynamic range of the system. Alternative start codons, such as GUG or UUG, can also be used, though AUG is the most common and generally leads to higher translation efficiency. The start codon (218), by defining the translation initiation site, plays a role in regulating protein production. Its precise location downstream of the output toehold sequester domain ensures that translation is activated only after successful strand exchange, minimizing leaky expression and optimizing the riboregulator's performance.

The n-terminal linker (219) is an RNA sequence that encodes for a short amino acid sequence located at the beginning of the translated protein. It is encoded by the RNA sequence immediately following the start codon (218) in the protein coding sequence (220). This sequence is typically designed to have very little secondary structure to promote high translation initiation rates. One benefit of the n-terminal linker is it can encode for protein sequences that enhance protein functionality. The linker can be designed to improve protein solubility, stability, or folding. It can also serve as a site for attaching functional tags, expanding the protein's utility in various applications. Variations in length and amino acid composition can be tailored to specific requirements. The n-terminal linker (219) provides a versatile tool for controlling translation rates and enhancing protein functionality. Its flexible design allows for the incorporation of various modifications or tags, improving protein stability, facilitating purification, or directing subcellular localization. This adaptability expands the utility of the translated protein in diverse applications.

The protein coding sequence (220) is the component of the THE riboregulator gate (225) that determines the amino acid sequence of the regulated protein. It is located downstream of the start codon (218) and n-terminal linker (219) on the gate prime strand (202). This sequence (220) includes a series of codons that specify the order of amino acids in the protein. One benefit of the protein coding sequence (220) is its modularity. Any gene of interest can be inserted downstream of the regulatory elements, allowing the THE riboregulator to control the expression of a wide range of proteins. This customization capability makes the system highly versatile and adaptable to various applications. Variations in codon usage can optimize translation efficiency in different organisms. The protein coding sequence (220) provides the modularity that allows the THE riboregulator to control the expression of any protein of interest. This flexibility expands its utility in diverse biological systems and enables customization for specific applications. The coding sequence's adaptability makes it a powerful tool for regulating gene expression.

The toehold spacer domain (221) is an optional RNA sequence located between the ribozyme sequence (209) and the gate input toehold domain (210). This spacer domain (221) can be used to further optimize the kinetics of strand exchange by providing additional space between the ribozyme and dsRNA gate, preventing potential steric hindrance during the strand exchange reaction. One benefit of the toehold spacer domain (221) is its ability to enhance strand exchange efficiency. The spacer provides flexibility to the RNA molecule, facilitating the interaction between the toehold domain and the input strand and preventing steric clashes that might hinder the reaction. Variations in length and sequence can be explored to optimize performance. The toehold spacer domain (221), by providing additional flexibility to the RNA molecule, contributes to the efficiency of the strand exchange reaction. It reduces the potential for steric hindrance, optimizing the interaction between the toehold and input strand and enhancing the overall performance of the THE riboregulator.

The distal (d) ribosome standby domain (222) is a sequence on the input strand (215) located upstream of the input branch migration domain (206), wherein the molecule is arranged or drawn from 3′ to 5′. This domain (222) does not directly participate in the strand exchange reaction but can influence the rate of protein translation in the ON state. One benefit of incorporating a distal ribosome standby domain is tunable protein expression. The length of this domain can be adjusted, typically from 0 to 20 bases, to modulate the rate of protein translation. Longer standby domains generally lead to faster protein production, providing a mechanism for fine-tuning the system's response. The distal (d) ribosome standby domain (222) allows for tunable control over protein expression levels. Its presence expands the design space for optimizing the system's performance in various applications.

The input toehold domain (223) is a single-stranded RNA sequence located at the 3′ end of the input strand (215). It is complementary to the gate input toehold domain (210) on the gate prime strand (202). This complementarity ensures specific recognition between the input strand (215) and the THE riboregulator gate (225), initiating the strand exchange reaction. The length and sequence of the input toehold domain (223) can be varied to optimize binding affinity and reaction kinetics. A primary benefit of this domain is specific activation. The input toehold domain (223), through its sequence complementarity, ensures that only the correct input strand can bind to the gate and initiate strand exchange, minimizing off-target effects and ensuring precise control over the regulatory process. The input toehold domain (223) initiates the regulatory process through its specific interaction with the gate input toehold domain. This specific recognition ensures that the THE riboregulator responds only to the intended input signal, minimizing off-target effects and maximizing control over gene expression.

The translation-regulated protein (224) is the protein product whose expression is controlled by the THE riboregulator (225). It is encoded by the protein coding sequence (220) on the gate prime strand (202). The expression of this protein is regulated by the strand exchange reaction. In the OFF state (225), the ribosome binding sequence (226) is sequestered, preventing translation. In the ON state (227), the RBS is exposed, allowing translation to proceed. One benefit of this arrangement is the ability to regulate the expression of any protein of interest. The modular design of the THE riboregulator allows the protein coding sequence (220) to be easily replaced, enabling control over a wide range of proteins and facilitating diverse applications. The translation-regulated protein (224) represents the output of the THE riboregulator system. Its controlled expression, mediated by the strand exchange reaction, enables precise regulation of protein levels in response to the input signal. The modularity offered by the protein coding sequence allows customization of the system to regulate the expression of any protein of interest.

The toehold exchange (THE) riboregulator gate (225), in its OFF state, is the initial configuration of the riboregulator before the strand exchange reaction. In this state, the output toehold domain (205) is bound to the ribosome binding sequence (226), preventing translation initiation. This configuration ensures tight control over protein expression, minimizing leaky expression in the absence of the input signal. One benefit of the OFF state configuration is its ability to maintain stringent control over protein production. The sequestration of the RBS prevents unintended protein expression, while the accessibility of the gate input toehold domain (210) allows for a rapid response to the presence of the input strand (215). This precise control and responsiveness are critical features of the THE riboregulator. The OFF state (225) of the THE riboregulator gate represents the initial, inactive configuration of the system. It ensures tight control over gene expression by sequestering the ribosome binding sequence and preventing translation initiation until the specific input signal is detected. This precise regulation is essential for controlling cellular processes and optimizing the system's performance.

The ribosome binding sequence (226) is a short RNA sequence found on the gate prime strand (202) that is essential for initiating protein translation. It serves as the binding site for the ribosome, the molecular machine responsible for protein synthesis. In the OFF state (225) of the THE riboregulator gate, the RBS is sequestered by the output toehold domain (205), preventing translation. Upon strand exchange and formation of the ON state (227), the RBS is exposed, allowing the ribosome to bind and initiate translation of the protein coding sequence (220). One benefit of this sequence is its essential role in regulated protein production. The RBS sequence and its interaction with the output toehold domain determine the level of translational control. Stronger RBS sequences typically lead to higher translation rates, while weaker sequences result in lower protein production. This controllability allows for fine-tuning of protein expression levels. Variations in the RBS sequence can be used to optimize translation efficiency. It should be appreciated that the output toehold sequester domain 213 is a sequence that is a genus to the ribosome binding sequence 226, and the ribosome binding sequence 226 is complementary to the ribosome binding sequence (RBS) sequestering domain 228. That is, the output toehold sequester domain 213 is the general term for this domain on ctRSD gates, and the ribosome binding sequence 226 is a specific type of sequence for use in a THE riboregulator.

The THE riboregulator gate (227), in its ON state, is the configuration of the riboregulator after the strand exchange reaction has occurred. In this state, the output strand (201) has been displaced by the input strand (215), and the ribosome binding sequence (226) is exposed. This exposure allows ribosomes to bind and initiate translation of the protein coding sequence (220), resulting in protein production. One benefit of the ON state configuration (227) is its direct coupling of RNA strand exchange to protein production. The exposure of the RBS enables a precise and readily measurable readout of the riboregulator's activity. This direct link between RNA computation and protein expression opens exciting possibilities for designing complex genetic circuits and synthetic biology applications.

The ribosome binding sequence (RBS) sequestering domain (228) is a short, single-stranded RNA sequence located between the input and output branch migration domains (206, 204) on the output strand (201). It serves to control whether the output strand (201) can react with other downstream components. If the output toehold domain is sequestered then the output strand of the gate cannot react downstream, if the output toehold domain is released due to strand exchange, the output strand can react downstream. This domain is typically 4 to 12 nucleotides long and its sequence is designed to be complementary to a region of the gate prime strand known as the ribosome binding sequence (226). One benefit of the ribosome binding sequence (RBS) sequestering domain 228 is its role in regulating the rate and specificity of an RNA strand exchange reaction, ensuring a rapid and controlled response to the input signal. The length and sequence of the toehold can be modified to adjust the kinetics of strand exchange, allowing for fine-tuning of the system's sensitivity and response time. This toehold-mediated initiation enhances the rate and specificity of the reaction, ensuring a rapid and controlled response to the presence of the input signal. It should be appreciated that the ribosome binding sequence (RBS) sequestering domain 228 is a sequence that is a species of the output toehold domain 205, and the ribosome binding sequence (RBS) sequestering domain 228 is complementary to the ribosome binding sequence 226. That is, the output toehold domain 205 is the general term for this domain on ctRSD gates, and the ribosome binding sequence (RBS) sequestering domain 228 is a specific type of sequence for use in a THE riboregulator.

The DNA sequence (300) serves as the blueprint for the THE riboregulator (225). It contains the genetic information that encodes the RNA sequences necessary for the riboregulator's function, including the gate input toehold domain (210), output toehold sequester domain (213), and self-cleaving ribozyme sequence (209). The DNA sequence is designed based on the desired functionality of the riboregulator, taking into account the specific input RNA to be recognized and the protein to be regulated. One benefit of encoding the riboregulator in a DNA sequence is its stability and ease of manipulation. DNA is a more stable molecule than RNA, making it easier to store, amplify, and modify. This allows for the creation of libraries of DNA sequences encoding different riboregulators, facilitating the development and optimization of new genetic control elements. Variations in the DNA sequence can be introduced to optimize the riboregulator's performance or to adapt it to different expression systems. The DNA sequence (300) provides a stable and readily manipulable template for generating the RNA components of the THE riboregulator. Its design flexibility enables the creation of customized riboregulators with specific functionalities and optimized performance characteristics.

The promoter sequence (301) is a specific DNA sequence located upstream of the sequences encoding the RNA components of the THE riboregulator (225). It serves as the binding site for RNA polymerase, the enzyme responsible for RNA transcription. The choice of promoter sequence (301) depends on the expression system used. Constitutive promoters drive continuous transcription, while inducible promoters allow for controlled expression under specific conditions. One benefit of the promoter sequence is regulated riboregulator production. The choice of promoter dictates the level and timing of riboregulator expression, enabling precise control over its production and optimizing its function in the desired context. Variations in promoter strength and inducibility can fine-tune expression levels. The promoter sequence (301) plays a critical role in regulating the production of the THE riboregulator, providing control over its expression level and timing. This regulated expression is crucial for optimizing riboregulator function and tailoring it to specific applications.

The sequence encoding the gate input toehold domain (302) is a segment of the DNA sequence (300) that specifies the RNA sequence of the gate input toehold domain (210). This DNA sequence is carefully designed to be complementary to the input toehold domain (223) of the input strand (215), ensuring specific recognition and initiation of the strand exchange reaction. One benefit of designing this sequence is programmable input specificity. The sequence of the gate input toehold domain determines which input RNA molecule can activate the riboregulator. This programmability enables the creation of customized riboregulators that respond to a wide range of input signals. Variations in sequence length and nucleotide composition can be used to fine-tune the affinity and kinetics of toehold binding. The sequence encoding the gate input toehold domain (302) provides a mechanism for programming input specificity, enabling the design of riboregulators that respond to specific RNA signals. This targeted activation minimizes off-target effects and enhances the controllability and precision of the system.

The sequence encoding the output toehold sequester domain (303) is a segment of the DNA sequence (300) that dictates the RNA sequence of the output toehold sequester domain (213). This domain is critical for regulating translation initiation. It is designed to be complementary to the ribosome binding sequence (226), ensuring efficient sequestration of the RBS in the OFF state (225) of the riboregulator. One benefit of this sequence is it provides stringent translational control. The sequence of the output toehold sequester domain determines its ability to effectively bind to and sequester the RBS. This sequestration prevents leaky protein expression in the absence of the input signal, maximizing the dynamic range of the riboregulator. Variations in sequence length and complementarity to the RBS can fine-tune the level of translational repression. The sequence encoding the output toehold sequester domain (303) ensures precise control over translation initiation by specifying a sequence that efficiently sequesters the ribosome binding sequence. This tight regulation minimizes background expression and enhances the dynamic range of the riboregulator, enabling precise control over protein production.

The sequence encoding the self-cleaving ribozyme sequence (304) is a part of the DNA sequence (300) that specifies the RNA sequence of the ribozyme (209). This sequence is essential for the efficient production of the functional THE riboregulator gate (225). It encodes a catalytic RNA molecule that can cleave itself, generating the gate input toehold domain (210) during transcription. One benefit of encoding a self-cleaving ribozyme is streamlined gate production. Co-transcriptional self-cleavage simplifies the synthesis process, eliminating the need for separate enzymatic processing steps. This improves the efficiency and robustness of riboregulator production, especially in cellular environments. Different ribozyme sequences, such as those derived from the HDV, hammerhead, or hairpin ribozyme, can be used depending on the desired cleavage activity and compatibility with the surrounding sequences. The inclusion of a sequence encoding a self-cleaving ribozyme (304) significantly improves the efficiency of THE riboregulator production. This self-processing feature simplifies the synthesis process, ensuring robust production of the functional gate and enhancing the overall controllability of the system.

The sequence encoding the start codon (305) determines the translation initiation site. This sequence within the DNA sequence (300) specifies the RNA sequence of the start codon (218), which is typically AUG. The precise placement of the start codon downstream of the regulatory elements ensures that protein translation only begins after the strand exchange reaction has occurred and the ribosome binding sequence (226) is exposed. One benefit of encoding the start codon is precise control of translation initiation. The location of the start codon relative to the RBS and the output toehold sequester domain (213) determines the stringency of translational control. This arrangement minimizes leaky expression in the OFF state (225) and allows for efficient protein production in the ON state (227). While AUG is the most common start codon, variations such as GUG or UUG can also be used, offering some flexibility in optimizing translation initiation. The sequence encoding the start codon (305) is crucial for regulating the timing and accuracy of protein translation. Its precise positioning ensures that translation occurs only after successful strand exchange, maximizing control over protein production and optimizing the riboregulator's performance.

The sequence encoding the c spacer domain (306) determines the length and composition of the spacer between the output toehold sequester domain (213) and the start codon (218). This spacer (217) plays a key role in optimizing translation efficiency. The length of the c spacer domain is typically between 5 and 7 nucleotides. One benefit of encoding the c spacer domain is enhanced translation efficiency. The spacer ensures proper spacing between the ribosome binding sequence (226) and the start codon, promoting efficient ribosome binding and maximizing protein production in the ON state (227) of the riboregulator. Variations in spacer length and sequence can further optimize translation rates, tailoring them to specific application requirements. The sequence encoding the c spacer domain (306), by specifying the optimal spacing between the RBS and the start codon, contributes significantly to enhancing translation efficiency. This improved efficiency maximizes protein production when the THE riboregulator is activated, optimizing the system's performance.

The sequence encoding the protein coding sequence (307) is the portion of the DNA sequence (300) that defines the amino acid sequence of the regulated protein (224). It is located downstream of the start codon (218) and consists of a series of codons that specify the order of amino acids in the protein. One benefit of this sequence is the ability to regulate any protein of interest. The modular design of the THE riboregulator allows the protein coding sequence (220) to be easily swapped, enabling researchers to control the expression of diverse proteins using the same regulatory mechanism. This adaptability makes the system versatile and applicable to a wide range of biological questions and genetic engineering applications. Variations in codon usage can optimize translation efficiency in different organisms.

The specific elements of the DNA sequence (300) each contribute to the precise and controlled production of the THE riboregulator. The promoter sequence (301) ensures regulated expression, while the sequences encoding the gate input toehold domain (302), output toehold sequester domain (303), and self-cleaving ribozyme sequence (304) define the core components of the regulatory mechanism. The sequences encoding the start codon (305), c spacer domain (306), and protein coding sequence (307) dictate the translation initiation site, optimize spacing, and determine the regulated protein's amino acid sequence. These elements enable precise control over gene expression and protein production.

The ribosome standby site (401) is a region on the THE riboregulator gate (227) that becomes accessible after the strand exchange reaction. It is located near the exposed ribosome binding sequence (226), and its accessibility can influence translation initiation. When the ribosome standby site (401) is available, it may facilitate more efficient ribosome binding to the nearby RBS, thereby enhancing translation initiation rates. The standby site may provide a platform for interactions with other cellular factors that promote translation. One benefit of the ribosome standby site is its potential to enhance translation efficiency. By providing a favorable environment for ribosome binding, it may boost protein production in the ON state (227). The specific sequence and structure of the ribosome standby site (401) can be optimized to maximize its positive effect on translation. The ribosome standby site (401) can play a supportive role in enhancing translation efficiency by providing a favorable context for ribosome binding to the exposed ribosome binding sequence. This may contribute to a more robust and efficient protein production when the THE riboregulator is activated.

The THE riboregulator system can be extended to incorporate multilayer cascades, enabling complex logic functions and sophisticated control over protein translation. In such cascades, the output of one THE riboregulator gate can serve as the input for another gate, creating a chain of regulatory events. This arrangement allows for the integration of multiple input signals and the implementation of Boolean logic functions, such as AND, OR, and NOT gates. One benefit of multilayer cascades is the ability to perform complex computations. By linking multiple THE riboregulators together, the system can process multiple RNA inputs and generate intricate expression patterns, mimicking the complexity of natural gene regulatory networks. It is contemplated that these could be THE riboregulators or ctRSD gates, which do not encode for a protein but can conduct strand exchange to release outputs. Variations in cascade design, such as the number of layers and the types of logic gates employed, allow for customization of the system's response and dynamic range. Multilayer cascades of THE riboregulators provide a platform for implementing sophisticated control circuits in synthetic biology. The ability to integrate multiple inputs, perform complex computations, and generate intricate expression patterns expands the potential of RNA-based genetic control systems for engineering cellular behavior and developing advanced biotechnological applications.

The THE riboregulator system's design allows for flexibility in the choice of ribozymes and toehold sequences. While the HDV ribozyme is commonly used for its efficient self-cleavage activity, other ribozymes, such as the Twister or Pistol ribozyme, could also be employed. Similarly, the sequences of the toehold domains can be customized to recognize different input RNA molecules, expanding the range of signals the system can respond to. One benefit of employing alternative ribozymes and toeholds is the adaptability of the THE riboregulator to different organisms and expression systems. Ribozymes and toeholds with varying activities and specificities can be chosen to optimize performance in different cellular contexts, ensuring compatibility and efficient riboregulator function. Further, orthogonal ribozymes and toeholds, which do not cross-react with each other, can be used to build more complex and independent control circuits within the same cell. The flexibility in ribozyme and toehold choice broadens the applicability of the THE riboregulator system. It allows implementers to tailor the system to different organisms and expression contexts, maximizing performance and enabling the construction of more sophisticated genetic circuits.

The lengths and complementarity of the various domains in the THE riboregulator, including the toehold, branch migration, and spacer domains, can be adjusted to fine-tune the system's performance. Toehold length influences the kinetics of strand exchange, with longer toeholds generally resulting in faster reactions. The length of the branch migration domain affects the stability of the RNA duplex and the rate of strand exchange. Spacer domains can be introduced to optimize spacing and prevent steric hindrance. One benefit of varying domain parameters is the precise control over riboregulator activity. Optimizing the lengths and complementarity of the domains allows for fine-tuning of the reaction kinetics, achieving specific activation thresholds, response times, and dynamic ranges. This precise control can be used for tailoring the system to particular applications and maximizing its effectiveness in regulating gene expression. The ability to vary domain parameters provides a mechanism for precise control over THE riboregulator function. Optimizing the length and complementarity of different domains enables customization of the system's kinetics, activation thresholds, and dynamic range, making it a versatile tool for regulating gene expression in diverse contexts.

The protein coding sequence (220) of the THE riboregulator can be modified to produce a wide range of output proteins. This modularity expands the system's utility in various applications. Fluorescent proteins can be used for visualizing gene expression and tracking cellular processes. Enzymes can be employed for metabolic engineering and biocatalysis. Growth factors can be used for therapeutic applications. One benefit of varying output protein sequences is customized functionality. By selecting specific protein coding sequences, researchers can tailor the output of the THE riboregulator to achieve desired cellular responses or functionalities. This customization expands the applicability of the system in various fields, including biotechnology, medicine, and synthetic biology. The ability to vary the output protein sequence makes the THE riboregulator a highly adaptable platform for controlling diverse cellular functions and implementing customized genetic circuits. This versatility expands its utility in various applications, from basic research to therapeutic interventions.

While the THE riboregulator gate (225) is primarily composed of RNA, variations in its composition can be considered. Modified nucleotides, such as those with altered base pairing properties or enhanced stability, can be incorporated into the RNA sequence. These modifications can enhance the riboregulator's resistance to degradation by cellular RNases, improve its binding affinity to the input strand, or modulate the kinetics of the strand exchange reaction. Furthermore, the RNA components of the gate can be conjugated to, or interact with, other molecules, such as proteins or small molecules, to add functionalities or to facilitate delivery into cells. For example, conjugating the riboregulator to a cell-penetrating peptide can enhance its uptake into cells, expanding its potential for in vivo applications.

The DNA sequence (300) that encodes the THE riboregulator can be synthesized using various methods. Chemical synthesis allows for precise control over the sequence and enables the incorporation of modified nucleotides or other chemical modifications. Enzymatic methods, such as polymerase chain reaction (PCR), can be used to amplify existing DNA templates or to generate variant sequences through mutagenesis. The choice of synthesis method depends on the specific application and the desired characteristics of the DNA sequence. The synthesized DNA can be incorporated into plasmids or other vectors for expression in cells or used directly in cell-free systems.

The promoter sequence (301) can be selected from a wide range of promoters with varying strengths and regulatory properties. Constitutive promoters, such as the T7 promoter, drive continuous gene expression. Inducible promoters, such as the lac operon promoter, allow for controlled expression in response to specific stimuli, such as the presence of lactose or IPTG. Tissue-specific promoters can restrict riboregulator expression to certain cell types or tissues. The choice of promoter sequence (301) is crucial for optimizing riboregulator expression and function in the desired context. Furthermore, synthetic promoters can be designed to incorporate specific regulatory elements or to achieve desired expression profiles.

The protein coding sequence (220) can encode a wide variety of proteins, expanding the utility of the THE riboregulator system. Fluorescent proteins, such as GFP or RFP, can be used as reporters to monitor gene expression levels. Enzymes can be employed for metabolic engineering or biocatalysis, allowing for the production of valuable compounds or the modification of cellular pathways. Therapeutic proteins, such as antibodies or growth factors, can be expressed under the control of the THE riboregulator for targeted therapies. The choice of protein coding sequence depends on the specific application and the desired outcome.

The gate input toehold domain (210) and output toehold (205) can range from 4 to 15 nucleotides in length, specifically from 6 to 12 nucleotides, and more specifically from 7 to 10 nucleotides. Longer input toeholds generally promote faster strand exchange kinetics. The optimal toehold length depends on the desired balance between reaction speed and specificity for a given application. The output toehold sequester domain (213), which is a ribosome binding sequence in a THE riboregulatory, typically ranging from 4 to 12 nucleotides. The c spacer domain (217) length influences translation initiation efficiency, generally ranging from 3 to 10 nucleotides, specifically from 4 to 8 nucleotides, and more specifically from 5 to 7 nucleotides. The protein coding sequence (220) can encode proteins of varying lengths, ranging from tens to thousands of amino acids, depending on the desired protein product.

Within the output strand (201), the input branch migration domain (206) can range from 10 to 50 nucleotides, specifically from 15 to 40 nucleotides, and more specifically from 10 to 20 nucleotides. The output branch migration domain (204) can have a similar length range. The output toehold domain (205) can range from 4 to 12 nucleotides, similar to the gate input toehold domain (210). The hairpin-forming sequence (203) can have a stem length ranging from 4 to 15 base pairs, with a loop size ranging from 3 to 10 nucleotides. The output wobble domain (207) can incorporate from 1 to 5 G-U wobble base pairs, and the linker (L) sequence (208) can range from 1 to 10 nucleotides.

On the gate prime strand (202), the substrate domain (211) can vary in length from 10 to 50 nucleotides, specifically from 15 to 40 nucleotides, and more specifically from 10 to 20 nucleotides, complementing the length of the input branch migration domain (206). The gate prime wobble domain (212) can include from 1 to 5 G-U wobble base pairings, similar to the output wobble domain. The output toehold sequester domain (213), complementary to the ribosome binding sequence (226), can range from 4 to 12 nucleotides. The transcription termination sequence (214) typically ranges from 30 to 80 nucleotides, depending on the RNA polymerase used.

In the input strand (215), the input toehold domain (223) can vary from 4 to 15 nucleotides, specifically from 6 to 12 nucleotides, and more specifically from 7 to 10 nucleotides, complementing the gate input toehold domain (210). The input branch migration domain (206) can range from 10 to 50 nucleotides, specifically from 15 to 40 nucleotides, and more specifically from 20 to 30 nucleotides. The distal ribosome standby domain (222), which modulates reaction kinetics, can range from 0 to 20 nucleotides in length. These diverse size ranges provide flexibility for optimizing the THE riboregulator's performance in various contexts.

While the hairpin-forming sequence (203) is typically designed to form a single hairpin structure, variations in its secondary structure can be considered. Multiple hairpins, internal loops, or bulges can be introduced to modulate the stability and accessibility of the output strand (201). The specific shape of the hairpin can influence its interaction with other RNA molecules or proteins, providing additional layers of control over riboregulator function. Circular RNA structures for the gate prime strand can also be employed, offering enhanced stability and resistance to exonucleases. These variations in shape and geometry expand the design space for optimizing riboregulator performance and tailoring it to specific applications.

The overall three-dimensional structure of the THE riboregulator gate (225) can be influenced by the lengths and sequences of its constituent domains. While a linear structure is commonly depicted, the gate can adopt various conformations depending on the presence of other RNA molecules or proteins. Tertiary interactions between different domains within the gate, or between the gate and other cellular components, can influence its activity and stability. Understanding the three-dimensional structure of the riboregulator can provide insights into its mechanism of action and guide the design of more efficient and specific genetic control elements. Computational modeling and structural analysis techniques, such as RNAfold and SHAPE-Seq, can be used to predict and validate riboregulator structures.

The input strand (215) can adopt various conformations depending on its sequence and environment. While typically considered a linear molecule, it can form secondary structures, such as hairpins or loops, which may influence its interaction with the THE riboregulator gate (225). The presence of RNA-binding proteins or other cellular factors can also influence the input strand's shape and accessibility. Understanding these structural variations is crucial for optimizing the strand exchange reaction and ensuring precise control over riboregulator activity.

While the interaction between the THE riboregulator gate (225) and the input strand (215) is typically described as a simple bimolecular reaction, more complex interactions can be envisioned. Multiple input strands can be designed to interact with the same gate, either cooperatively or competitively. Cooperative binding could enhance the sensitivity of the riboregulator, allowing it to respond to lower concentrations of input signals. Competitive binding could implement logic functions, such as an AND gate where two distinct input strands are required for activation. Further, the output strand (201) can be designed to interact with other RNA molecules or proteins, expanding the downstream effects of riboregulator activation. For instance, the output strand could activate a second riboregulator gate or modulate the activity of a cellular enzyme.

The self-cleaving ribozyme sequence (209) can be designed to interact with other cellular components to further control riboregulator production or activity. For instance, the ribozyme's activity could be modulated by the presence of a specific small molecule or protein, creating an additional layer of regulation. Alternatively, the cleaved ribozyme could function as a signaling molecule, triggering downstream events or interacting with other cellular components. The protein coding sequence (220) can be engineered to produce proteins with multiple functional domains or to interact with other proteins in a complex. This can expand the range of cellular functions controlled by the THE riboregulator, enabling more sophisticated manipulation of cellular behavior. For example, the regulated protein could be a transcription factor that controls the expression of multiple genes, creating a cascade of regulatory events.

In an embodiment, a process for making a toehold exchange (THE) riboregulator (225) comprises: (a) designing a DNA sequence (300) that encodes for the THE riboregulator (225); (b) obtaining the DNA sequence (300); and (c) transcribing the DNA sequence (300) to produce the THE riboregulator (225). In an embodiment, the DNA sequence (300) comprises: a promoter sequence (301) for an RNA polymerase; a sequence (302) that encodes for a gate input toehold domain (210); a sequence (303) that encodes for an output toehold sequester domain (213); and a sequence (304) that encodes for a self-cleaving ribozyme sequence (209). In an embodiment, the sequence (304) is positioned downstream of sequence (302) and upstream of sequence (303). In an embodiment, the DNA sequence (300) further comprises a sequence (305) that encodes for a start codon (218); a sequence (306) that encodes for a c spacer domain (217); and a sequence (307) that encodes for a protein coding sequence (220). In an embodiment, sequence (306) is positioned downstream of sequence (303) and upstream of sequence (305), and sequence (307) is positioned downstream of sequence (305). In an embodiment, the step of obtaining the DNA sequence (300) comprises synthesizing the DNA sequence (300). In an embodiment, the step of transcribing comprises conducting transcription in a cell-free expression system. In an embodiment, the process further comprises translating the protein coding sequence (220). In an embodiment, the process further comprises measuring performance of the THE riboregulator (225). In an embodiment, the THE riboregulator (225) is transcribed in the presence of a complementary input strand (215) to produce a translation-regulated protein (224).

The process of making a THE riboregulator (225) begins with designing a DNA sequence (300) that encodes the riboregulator. This involves specifying the nucleotide sequence that will be transcribed into the functional RNA molecule. Software tools, such as NUPACK, can be used to design sequences that fold into desired secondary structures and minimize unintended interactions. The design process considers the sequences of the toehold domains, branch migration domains, and other functional elements to ensure proper riboregulator function. One benefit of this step is precise control over the riboregulator's structure and function. By carefully designing the DNA sequence, researchers can tailor the riboregulator's response to specific input signals and control the expression of the desired protein. Variations in sequence design can be used to optimize the riboregulator's performance, kinetics, and dynamic range.

The next step in the process is obtaining the DNA sequence (300). This can be achieved through chemical synthesis, where the DNA sequence is built nucleotide by nucleotide. Alternatively, enzymatic methods, such as PCR, can be used to amplify existing DNA templates. One benefit of obtaining the designed DNA sequence is that it provides the physical template necessary for RNA production. This step bridges the gap between the digital design and the physical creation of the riboregulator. The method used to obtain the DNA depends on the length and complexity of the sequence, as well as the available resources and expertise.

The final step is transcribing the DNA sequence (300) to produce the THE riboregulator (225). This involves using an RNA polymerase enzyme to synthesize an RNA molecule that is complementary to the DNA template. Transcription can be performed in vitro using purified enzymes or in vivo within cells. One benefit of this step is the generation of the functional RNA molecule. Transcription produces the RNA components of the riboregulator that will participate in the strand exchange reaction and control protein translation. The choice of transcription method depends on the desired scale of production and the intended application of the riboregulator.

The design of the DNA sequence allows precise control over the structure and function of the THE riboregulator. Obtaining the DNA provides the physical template for RNA synthesis. Transcribing the DNA sequence generates the functional RNA molecule. These steps ensure the accurate and efficient production of the riboregulator, enabling researchers to control gene expression with high precision and versatility.

The DNA sequence (300) can include various elements that contribute to the functionality of the THE riboregulator (225). A promoter sequence (301) is a DNA sequence that initiates transcription of a gene. The promoter sequence (301) recruits RNA polymerase, which then transcribes the downstream sequences encoding the RNA components of the riboregulator. The choice of promoter influences the level and timing of riboregulator expression. The sequence encoding the gate input toehold domain (302) specifies the RNA sequence that will initiate strand exchange with the input strand (215). The sequence encoding the output toehold domain (303) determines the sequence that will sequester the ribosome binding sequence (226) in the OFF state (225) of the riboregulator. The sequence encoding the self-cleaving ribozyme sequence (304) specifies the ribozyme responsible for generating the gate input toehold domain (210) during transcription. The inclusion of these sequences ensures the precise and controlled production of the functional RNA components of the riboregulator.

Positioning the sequence encoding the self-cleaving ribozyme sequence (304) downstream of the sequence encoding the gate input toehold domain (302) and upstream of the sequence encoding the output toehold sequester domain (303) ensures the correct order of RNA domains in the transcribed molecule. This arrangement facilitates proper folding and function of the riboregulator.

The DNA sequence (300) may further comprise sequences encoding additional elements of the THE riboregulator (225). The sequence encoding the start codon (305), typically AUG, defines the translation initiation site for the regulated protein (224). The sequence encoding the c spacer domain (306) specifies the spacer region between the output toehold sequester domain (213) and the start codon (218), optimizing translation initiation efficiency. The sequence encoding the protein coding sequence (307) determines the amino acid sequence of the regulated protein. These sequences expand the functionality and controllability of the riboregulator.

Positioning the sequence encoding the c spacer domain (306) downstream of the sequence encoding the output toehold sequester domain (303) and upstream of the sequence encoding the start codon (305), with the sequence encoding the protein coding sequence (307) downstream of the start codon sequence, ensures the correct order of elements in the transcribed RNA molecule. This arrangement facilitates proper ribosome binding, translation initiation, and protein production.

Synthesizing the DNA sequence (300) offers precise control over its nucleotide composition, enabling researchers to incorporate specific sequences for toeholds, branch migration domains, ribozymes, and protein coding regions. This customized synthesis approach allows for the creation of riboregulators with tailored functionalities and optimized performance characteristics.

Conducting transcription in a cell-free expression system offers advantages for producing the THE riboregulator (225). Cell-free systems provide a controlled environment for RNA synthesis, allowing for manipulation of reaction conditions and incorporation of modified nucleotides or other chemical modifications. This approach facilitates the production of riboregulators with enhanced stability or altered functionality.

Translating the protein coding sequence (220) in a cell-free system allows for direct observation of riboregulator function and measurement of protein production. This provides a valuable tool for characterizing the performance of the riboregulator and optimizing its design.

Measuring the performance of the THE riboregulator (225) is essential for assessing its activity and optimizing its design. Various assays, such as fluorescence or luminescence-based reporter assays, can be used to quantify protein production levels and determine the dynamic range of the riboregulator. These measurements guide the optimization of sequence parameters and ensure the riboregulator functions as intended.

Transcribing the THE riboregulator (225) in the presence of the complementary input strand (215) allows for assessment of the system's functionality in a controlled setting. Measuring the levels of the translation-regulated protein (224) produced under these conditions provides insights into the efficiency and kinetics of strand exchange and its effect on protein translation.

The inclusion of specific sequences in the DNA sequence (300), such as the promoter (301), sequences for functional domains (302, 303, 304), and sequences for translation elements (305, 306, 307), enables the creation of a functional THE riboregulator. The precise arrangement of these sequences ensures proper riboregulator assembly and activity. Synthesizing the DNA and performing transcription and translation in controlled environments, such as cell-free systems, facilitate characterization and optimization. Measuring the performance of the riboregulator and observing its response to the input strand provide valuable data for validating its functionality and guiding further improvements. These elements contribute to the robust and controlled production of a functional THE riboregulator for regulating protein translation.

Following DNA synthesis, the DNA sequence (300) encoding the THE riboregulator (225) can be amplified using polymerase chain reaction (PCR). PCR is a widely used technique for generating multiple copies of a specific DNA sequence. This amplification step is particularly useful when the initial amount of synthesized DNA is limited. The amplified DNA can then be cloned into a plasmid or other vector for downstream applications. Cloning involves inserting the DNA sequence into a vector, which is a DNA molecule that can replicate independently in a host cell. This facilitates the production of larger quantities of the DNA and enables its introduction into cells for in vivo expression of the THE riboregulator. Different cloning strategies, such as restriction enzyme cloning or Gibson assembly, can be employed depending on the vector and the desired application. PCR amplification and cloning provide efficient methods for generating large quantities of the DNA sequence encoding the THE riboregulator, facilitating its use in various experimental and application settings. Cloning into expression vectors enables controlled production of the riboregulator within cells, while direct use of amplified DNA is suitable for cell-free systems.

The THE riboregulator (225) can be expressed in vivo within cells by introducing the DNA sequence (300) into an appropriate expression vector and transfecting or transforming the vector into the chosen host cells. Expression vectors contain regulatory elements, such as promoters and terminators, that drive transcription and translation of the encoded gene. Different expression systems, including bacterial and other cells, can be used depending on the desired application and the characteristics of the protein being regulated. Following expression, the THE riboregulator can be purified from the cells using various methods, such as affinity chromatography or gel electrophoresis. Purification removes other cellular components and yields a concentrated preparation of the riboregulator for downstream applications. In vivo expression allows for the production of large quantities of the THE riboregulator within a cellular context. Purification ensures that the riboregulator is free from other cellular components, enabling its use in various applications, including in vitro assays, in vivo gene regulation studies, and therapeutic interventions.

The THE riboregulator (225) can be integrated into more complex genetic circuits, enabling sophisticated control over cellular processes. The output of the riboregulator can be linked to the expression of other genes, creating cascades of regulatory events. For instance, the output strand (201) can be designed to activate a second riboregulator, leading to the expression of a downstream gene. Alternatively, the regulated protein (224) could itself be a transcription factor that controls the expression of other genes. This modular design allows for the construction of complex genetic circuits with defined logic functions and dynamic responses. The integration of THE riboregulators into genetic circuits extends the functionality of these regulatory elements. The ability to link riboregulator activity to the expression of downstream genes or to connect multiple riboregulators in a cascade enables sophisticated control over cellular processes and opens possibilities for building complex synthetic biology systems with predictable behavior.

The THE riboregulator system has potential applications in both diagnostics and therapeutics. In diagnostics, the riboregulator can be designed to detect specific RNA molecules, such as those associated with viral infections or cancer. The presence of the target RNA would activate the riboregulator, triggering the expression of a reporter gene, such as a fluorescent protein or an enzyme that produces a colorimetric signal. In therapeutics, the riboregulator can be used to control the expression of therapeutic proteins, such as antibodies or growth factors. By targeting the riboregulator to specific cells or tissues, therapeutic protein expression can be localized and controlled, enhancing its efficacy and minimizing side effects. The THE riboregulator's ability to detect specific RNA molecules and control protein translation makes it a promising tool for developing new diagnostic and therapeutic applications. Its programmable nature and precise control over gene expression offer unique advantages for customizing these applications to different diseases and targets.

Designing a DNA sequence (300) that encodes for the THE riboregulator (225) can be an initial step in the process of making the riboregulator. This involves specifying the precise order of nucleotides that will be transcribed into the functional RNA molecule. The design process takes into account the desired secondary structure of the RNA, the sequences of the toehold domains (210, 223), the branch migration domains (204, 206), the ribosome binding sequence (226), the start codon (218), and the protein coding sequence (220). Software tools, such as NUPACK, can assist in the design process by predicting RNA secondary structure and identifying potential interactions between different domains. One benefit of this design step is precise control over the structure and function of the THE riboregulator. By carefully designing the DNA sequence, researchers can tailor the riboregulator's response to specific input signals and control the expression of the desired protein. Variations in sequence design, such as the length and sequence of the toehold domains, can be introduced to optimize the kinetics and thermodynamics of the strand exchange reaction. The careful design of the DNA sequence (300) is essential for creating a functional THE riboregulator (225). This step provides a blueprint for the RNA molecule, enabling researchers to control its structure, function, and interactions with other components of the system. This precise control is crucial for achieving desired regulatory outcomes and maximizing the effectiveness of the riboregulator.

Obtaining the DNA sequence (300) is the next step in producing the THE riboregulator (225). After the DNA sequence has been designed, it can be physically obtained to serve as a template for RNA transcription. There are several methods for obtaining the DNA sequence, and one approach is chemical synthesis, where the DNA sequence is built nucleotide by nucleotide using automated synthesizers. This method allows for precise control over the sequence and enables the incorporation of modified nucleotides or other chemical modifications. Another method is enzymatic synthesis, such as PCR amplification, which is particularly useful when a small amount of starting DNA template is available. PCR generates multiple copies of the target sequence, providing sufficient material for downstream applications. One benefit of obtaining the DNA sequence is the availability of a physical template for RNA production. This step bridges the gap between the digital design and the physical creation of the THE riboregulator. The choice of method depends on the length and complexity of the sequence, the available resources and expertise, and the desired scale of production. Obtaining the DNA sequence (300) provides the essential physical template for generating the RNA components of the THE riboregulator (225). Whether through chemical synthesis or enzymatic amplification, this step brings the designed sequence to life, enabling its use in downstream applications. The flexibility in obtaining the DNA sequence allows researchers to adapt the process to their specific needs and resources.

Transcribing the DNA sequence (300) involves the synthesis of an RNA molecule that is complementary to the DNA template. The process is carried out by an RNA polymerase enzyme, which binds to the promoter sequence (301) on the DNA template and initiates RNA synthesis. Transcription can be performed in vitro, using purified enzymes and controlled reaction conditions, or in vivo, by introducing the DNA sequence into cells. One benefit of transcribing the DNA sequence is the production of the functional RNA molecule that forms the core of the THE riboregulator. This RNA molecule contains the toehold domains, branch migration domains, and other sequences necessary for the strand exchange reaction and regulation of protein translation. The choice of transcription method depends on the scale of production, the desired modifications to the RNA, and the intended application. In vitro transcription allows for greater control over reaction conditions and incorporation of modified nucleotides. In vivo transcription within cells provides a more natural context for riboregulator production. The transcription of the DNA sequence (300) marks the final step in the production of the functional THE riboregulator (225). This step generates the RNA molecule that will interact with the input strand (215) and control protein translation. The ability to perform transcription in vitro or in vivo provides flexibility and control over the production process.

Translating the protein coding sequence (220) involves decoding the mRNA sequence transcribed from the DNA sequence (300) into a polypeptide chain, forming the functional protein. This process occurs within cells or in cell-free systems and is carried out by ribosomes, the molecular machines responsible for protein synthesis. One benefit of including this translation step is production of the desired protein. Translation brings the genetic information encoded in the DNA to life, generating the functional protein that is regulated by the THE riboregulator. This process is essential for realizing the riboregulator's function and demonstrating its ability to control protein expression. Variations in translation conditions, such as temperature, magnesium concentration, and the presence of translation factors, can be used to optimize protein production yields. The translation of the protein coding sequence (220) is the culmination of the THE riboregulator's function, resulting in the production of the desired protein. This step confirms the riboregulator's ability to control protein expression and demonstrates its utility in various applications.

Measuring the performance of the THE riboregulator (225) is a crucial step in assessing its activity, optimizing its design, and validating its functionality. This typically involves quantifying the levels of the translation-regulated protein (224) produced under different conditions, such as in the presence or absence of the input strand (215). One benefit of measuring riboregulator performance is verification and optimization. These measurements provide valuable data for assessing the riboregulator's dynamic range, sensitivity, and response time. This information can then be used to guide the optimization of sequence parameters, such as toehold length and branch migration domain complementarity, to improve the riboregulator's performance and tailor it to specific applications. Various assays, including fluorescence-based reporter assays, enzyme activity assays, and Western blotting, can be used to quantify protein levels. The choice of assay depends on the nature of the regulated protein and the available detection methods. Measuring the performance of the THE riboregulator (225) is useful for validating its functionality and optimizing its design. This quantitative assessment provides valuable insights into the system's activity and guides improvements for achieving desired regulatory outcomes. One example involves the use of RT-qPCR to measure ribozyme cleavage for the RNA that make up THE riboregulator.

Transcribing the THE riboregulator (225) in the presence of a complementary input strand (215) and observing the production of the translation-regulated protein (224) demonstrate the functionality of the entire system. This step simulates the riboregulator's intended use and confirms its ability to control protein expression in response to the input signal. One benefit of this combined transcription and translation assay is validation of the complete system. It demonstrates the interplay between the riboregulator, the input strand, and the protein production machinery. By quantifying the amount of protein produced in the presence of the input strand, the efficiency of strand exchange, the level of translational control, and the overall dynamic range of the riboregulator can be assessed. This comprehensive evaluation is crucial for optimizing the system's performance and ensuring its effectiveness in regulating gene expression.

The inclusion and strategic arrangement of these elements and steps are essential for constructing a functional THE riboregulator (225). The promoter (301) drives regulated expression, the coding sequences for functional domains (302, 303, 304) determine the mechanism of action, and the coding sequences for translation elements (305, 306, 307) control protein production. Synthesizing, amplifying, and cloning the DNA sequence (300) provide the physical material for riboregulator production. Transcribing and translating the sequence generate the functional RNA and protein components. Measuring performance validates functionality and guides optimization. The combination of these precisely designed elements and carefully executed steps results in a robust and controllable system for regulating protein translation.

The process of transcribing the DNA sequence (300) can be performed under a variety of conditions, each optimized for different applications. In vitro transcription reactions typically involve purified RNA polymerase, a DNA template, and ribonucleotide triphosphates (rNTPs). Reaction temperature, reagent concentration (e.g., magnesium), and the presence of other cofactors can be adjusted to control transcription efficiency and RNA yield. For in vivo transcription, the DNA sequence (300) is introduced into cells, and cellular machinery carries out the transcription process. The choice of expression system, such as bacterial, yeast, or mammalian cells, influences the rate and fidelity of transcription. Further, the use of inducible promoters allows for controlled expression of the THE riboregulator (225) in response to specific stimuli.

The translation of the protein coding sequence (220) can be performed in vitro using cell-free expression systems or in vivo within cells. Cell-free systems provide a controlled environment for protein synthesis, allowing for manipulation of reaction parameters and incorporation of non-natural amino acids or other modifications. In vivo translation occurs within the complex environment of the cell, providing a more natural context for protein folding and function. The choice of translation system depends on the specific application and the characteristics of the protein being expressed. Optimization of translation conditions, such as temperature, magnesium concentration, and the presence of translation factors, enhances protein production efficiency.

In an embodiment, a process for regulating protein translation (400) with a toehold exchange (THE) riboregulator (225) comprises: (a) providing a THE riboregulator gate (225); and (b) contacting the THE riboregulator gate (225) with an input strand (215). In an embodiment, the THE riboregulator gate (225) comprises: a gate input toehold domain (210); an output toehold sequester domain (213) comprising a ribosome binding sequence 226; a start codon (218); a c spacer domain (217) positioned between domain (213) and the start codon (218); a protein coding sequence (220); and a self-cleaving ribozyme sequence (209). In an embodiment, the self-cleaving ribozyme sequence (209) produces the toehold exchange (THE) riboregulator gate (OFF) (225). In an embodiment, the input strand (215) comprises a distal (d) ribosome standby domain (222) and an input toehold domain (223). In an embodiment, the distal (d) ribosome standby domain (222) is 0 to 20 bases in length. In an embodiment, the process further comprises producing a strand exchange product (216) wherein the input strand (215) hybridizes to the gate prime strand (202) and displaces the output strand (201) from the THE riboregulator gate (225). In an embodiment, the strand exchange product (216) comprises a THE riboregulator gate (227) in an ON state (227). In an embodiment, the ribosome standby site (401) is exposed in the THE riboregulator gate (227). In an embodiment, the process further comprises initiating translation of the protein coding sequence (220) to produce a translation-regulated protein (224). In an embodiment, the contacting step occurs in a cell-free expression system, in a cell lysate, or in a cell.

The process of regulating protein translation (400) using a THE riboregulator (225) includes providing a THE riboregulator gate (225). This gate is a double-stranded RNA molecule designed to control protein translation. It contains sequences that are complementary to the input strand (215) and sequences that sequester the ribosome binding sequence (226). One benefit of providing the gate is that it establishes the foundation for regulated protein expression. The gate functions as a molecular switch, controlling the accessibility of the RBS and thereby determining whether translation can occur. The specific design of the gate, incorporating toeholds and branch migration domains, enables precise control over the strand exchange reaction and, consequently, protein production. The gate can be produced in vitro using enzymatic or chemical methods or expressed in vivo within cells.

The second step involves contacting the THE riboregulator gate (225) with an input strand (215). The input strand is a single-stranded RNA molecule designed to interact with the gate. Its sequence is complementary to a region on the gate, enabling specific recognition and binding. One benefit of this contacting step is it triggers the regulatory process. The interaction between the input strand and the gate initiates the strand exchange reaction. This reaction leads to the exposure of the RBS, thereby modulating protein translation. The input strand can be introduced into the system by various methods, including direct addition to an in vitro reaction or expression within cells.

The provision of the THE riboregulator gate establishes the molecular switch for controlling translation, while contacting the gate with the input strand triggers the regulatory process. The specific design of these components and their interaction provide a precise and programmable mechanism for regulating protein expression.

The THE riboregulator gate (225) comprises several functional domains that work together to control protein translation (400). The gate input toehold domain (210) is a single-stranded RNA sequence that initiates strand exchange by binding to the input toehold domain (223) of the input strand (215). The RBS (226) is sequestered by the output toehold domain (205) when the gate is in the OFF state (225). The start codon (218), usually AUG, marks the beginning of the protein coding sequence (220). The c spacer domain (217) is a short sequence between the RBS (226) and the start codon (218) that optimizes translation initiation. The protein coding sequence (220) specifies the amino acid sequence of the protein being regulated. The self-cleaving ribozyme sequence (209) produces a dsRNA gate suitable for strand exchange and generates the gate input toehold domain (210) during transcription. These elements ensure precise control over the timing and level of protein production.

The self-cleaving ribozyme sequence (209) plays a critical role in generating the dsRNA gate and the gate input toehold domain (210), which is essential for initiating the strand exchange reaction. This autocatalytic process simplifies riboregulator production and enhances its efficiency.

The input strand (215) comprises an input toehold domain (223), an input branch migration domain (206) and a distal (d) ribosome standby domain (222). The distal ribosome standby domain (222) is a sequence that can modulate the kinetics of strand exchange, while the input toehold domain (223) is essential for initiating the reaction by binding to the gate input toehold domain (210). These domains provide additional control over the timing and efficiency of riboregulation. Longer standby domains generally lead to faster protein production.

A distal (d) ribosome standby domain (222) length of 0 to 20 bases provides flexibility in tuning the kinetics of strand exchange and protein translation initiation rate. Shorter domains generally lead to faster kinetics and translation initiation.

Producing a strand exchange product (216) involves the hybridization of the input strand (215) to the gate prime strand (202), displacing the output strand (201) and exposing the ribosome binding sequence (226). This transition from the OFF state (225) to the ON state (227) is essential for activating protein translation.

The strand exchange product (216) comprises a THE riboregulator gate (227) in the ON state, with the ribosome binding sequence exposed, enabling translation initiation and protein production. This state represents the active configuration of the riboregulator.

The exposed ribosome standby site (401) on the THE riboregulator gate (227) in the ON state may provide a favorable context for ribosome binding and translation initiation, potentially enhancing translation efficiency by increasing the local concentration of ribosomes near the RBS.

Initiating translation of the protein coding sequence (220) involves the binding of ribosomes to the exposed ribosome binding sequence (226), leading to the synthesis of the translation-regulated protein (224). This step is the culmination of the riboregulatory process, resulting in the controlled production of the desired protein.

The contacting step between the THE riboregulator gate (225) and the input strand (215) can occur in various environments, including cell-free expression systems, cell lysates, or within living cells. Cell-free systems offer controlled reaction conditions, while cell lysates and living cells provide a more natural context for riboregulation.

The specific domains within the THE riboregulator gate (225), including the gate input toehold domain (210), the RBS sequestering domain (228) which is part of output strand (201), start codon (218), c spacer domain (217), protein coding sequence (220), and the self-cleaving ribozyme sequence (209), are essential for its function. The inclusion of the distal ribosome standby domain (222) and input toehold domain (223) on the input strand (215) provides additional control over the kinetics and specificity of the reaction. The formation of the strand exchange product (216) and the exposure of the ribosome binding sequence (226) and ribosome standby site (401) are crucial steps in activating protein translation. The initiation of translation and the choice of reaction environment further contribute to the controlled and efficient production of the translation-regulated protein (224). These elements together provide a robust and versatile platform for regulating protein expression.

The rate of protein translation (400) can be modulated by adjusting various parameters of the THE riboregulator (225) system. Changing the length of the distal ribosome standby domain (222) on the input strand (215) can fine-tune the kinetics of strand exchange, influencing the speed of the reaction and the timing of protein production. Modifying the complementarity between the toehold domains (210, 223) can also adjust the rate of strand exchange. Stronger complementarity generally leads to faster kinetics, while weaker complementarity slows down the reaction. Further, variations in the ribosome binding sequence (226) can affect translation initiation rates. Stronger RBS sequences typically result in higher translation rates, while weaker RBS sequences reduce protein production. Longer distal (d) ribosome standby domains (222) generally lead to faster translation initiation.

The activity of the THE riboregulator (225) can be influenced by environmental factors, such as temperature and ionic strength. Adjusting these parameters can modulate the stability of the RNA structures and the kinetics of strand exchange. Higher temperatures may destabilize RNA duplexes, potentially affecting the efficiency of toehold binding and branch migration. Changes in ionic strength can also influence RNA folding and stability, indirectly affecting riboregulator function. Optimizing these conditions can fine-tune the system's performance and adapt it to different environments or applications.

The THE riboregulator (225) can be integrated with other regulatory mechanisms to achieve more complex control over protein expression. For example, the input strand (215) could be produced under the control of a separate regulatory element, such as a small molecule-responsive riboswitch or a protein-binding aptamer. This would create a system where protein translation is regulated by both the input strand and the secondary regulatory element, providing an additional layer of control. Alternatively, the regulated protein (224) could itself be a component of a larger signaling pathway, influencing downstream cellular processes or interacting with other regulatory proteins.

For in vivo applications, the THE riboregulator system (225) can be delivered into cells using various methods. Viral vectors, such as lentiviruses or adenoviruses, can efficiently deliver the DNA or RNA components of the riboregulator into cells. Non-viral methods, such as lipid nanoparticles or electroporation, can also be used. The choice of delivery method depends on the target cell type, the efficiency of delivery required, and the potential for toxicity or immunogenicity. Once inside the cell, the THE riboregulator can function autonomously or be integrated into the cell's genome for stable expression.

Various methods can be employed to detect and quantify protein production controlled by the THE riboregulator (225). For reporter proteins, such as fluorescent proteins or luciferases, fluorescence or luminescence measurements provide a direct readout of protein expression levels. Enzyme activity assays can be used for regulated enzymes, providing a functional measure of riboregulator activity. Western blotting, ELISA, and other immunoassays can be used to quantify the amount of regulated protein produced. The choice of detection method depends on the nature of the regulated protein and the available detection technologies.

Producing a strand exchange product (216) is the central event in the THE riboregulator's (225) mechanism of action. It involves the displacement of the output strand (201) from the gate prime strand (202) by the input strand (215). This displacement occurs through a strand exchange reaction initiated by the hybridization of the input toehold domain (223) to the gate input toehold domain (210). One benefit of producing the strand exchange product (216) is the activation of protein translation. The displacement of the output strand exposes the ribosome binding sequence (226), allowing ribosomes to bind and initiate translation of the protein coding sequence (220). This transition from the OFF state to the ON state (227) is essential for regulating protein expression in response to the input signal. The stability of the strand exchange product can be influenced by the design of the toehold and branch migration domains, providing a mechanism for tuning the system's response. The formation of the strand exchange product (216) is a pivotal event in the regulation of protein translation. It signifies the transition of the THE riboregulator (225) from an inactive to an active state, enabling protein production in response to the input signal.

Initiating translation of the protein coding sequence (220) is the culmination of the THE riboregulator's (225) function. When the gate is in the ON state (227), the exposed ribosome binding sequence (226) allows ribosomes to bind and initiate translation of the protein coding sequence (220). This results in the production of the translation-regulated protein (224). One benefit of this step is the controlled production of the desired protein. The precise regulation of translation initiation by the THE riboregulator allows for fine-tuning of protein levels in response to specific input signals. This controllability is essential for optimizing cellular processes and implementing complex regulatory circuits. Variations in translation conditions, such as temperature, magnesium concentration, and the availability of translation factors, can be optimized to enhance protein production yields. Initiating translation of the protein coding sequence (220) is the final step in the regulatory process, resulting in the controlled production of the translation-regulated protein (224). This precise control over protein expression is crucial for achieving desired cellular responses and implementing complex genetic circuits.

The contacting step between the THE riboregulator gate (225) and the input strand (215) can be carried out under various conditions. In vitro reactions typically involve mixing the RNA molecules in a buffer solution with controlled pH, temperature, and ionic strength. The concentrations of the RNA molecules can be adjusted to optimize the kinetics and efficiency of strand exchange. For in vivo applications, the contacting step occurs within the cellular environment. The expression levels of the riboregulator and input strand can be controlled using appropriate promoters and regulatory elements. Further, the cellular environment itself, including the presence of RNA-binding proteins and other cellular factors, can influence the interaction between the riboregulator and the input strand. The choice of environment for the contacting step provides flexibility in studying and applying the THE riboregulator system. Cell-free systems, cell lysates, and living cells each offer unique advantages for investigating the system's function and adapting it to different applications, from basic research to therapeutic interventions.

The process for regulating protein translation (400) can be adapted to various applications by selecting appropriate conditions for the contacting step. In diagnostics, the reaction can be performed in a controlled in vitro setting, using defined concentrations of the riboregulator and input strand. The detection of the translated protein (224) can be coupled to a signal amplification mechanism, such as fluorescence or luminescence, to enhance sensitivity. In therapeutic applications, the riboregulator and input strand can be delivered into cells, and the contacting step occurs within the cellular environment. The controlled expression of the regulated protein (224) can then exert its therapeutic effect. For research purposes, the contacting step can be performed in cell lysates or other in vitro systems to study the kinetics and dynamics of the strand exchange reaction and its effects on protein translation. The flexibility in choosing reaction conditions and environments allows researchers to adapt the process to different applications and experimental settings.

In an embodiment, a process for regulating protein translation with a toehold exchange (THE) riboregulator (225) comprises: (a) providing a THE riboregulator gate (225) comprising: an output strand (201) comprising specified RNA sequences in sequential order, wherein certain domains are partially complementary to an input strand (215) and an output toehold domain (205) is partially complementary to an output toehold sequester domain (213); and a gate prime strand (202) hybridized to the output strand (201) comprising specified RNA sequences in sequential order, wherein a domain (210) is partially complementary to the input strand (215), a domain (211) is partially complementary to the input branch migration domain (206) of the output strand (201), a domain (213) is partially complementary to a ribosome binding sequence (RBS) (226), and certain domains promote translation of a protein coding sequence (220); and (b) contacting the THE riboregulator gate (225) with an input strand (215) comprising specified RNA sequences in sequential order, wherein a domain (223) is complementary to domain (210) of the gate prime strand (202) and a domain (206) is complementary to domain (211) of the gate prime strand (202); (c) initiating a strand exchange reaction between the THE riboregulator gate (225) and the input strand (215); (d) displacing the output strand (201) from the gate prime strand (202), thereby forming a strand exchange product (216); and (e) initiating translation of the protein coding sequence (220) at the exposed ribosome binding sequence (226) to produce a translation-regulated protein (224). In an embodiment, the self-cleaving ribozyme sequence (209) is a hepatitis delta virus (HDV) ribozyme. In an embodiment, the gate prime wobble domain (212) is connected to the substrate domain (211) through guanine-uracil (G-U) wobble base pairings. In an embodiment, the input branch migration domain (206) is connected to the output wobble domain (207) through G-U wobble base pairings. In an embodiment, the output toehold sequester domain (213) is a sequence complementary to a bacterial ribosome binding sequence. In an embodiment, the start codon (218) is AUG, GUG, or UUG. In an embodiment, the c spacer domain (217) is 5 to 7 bases in length. In an embodiment, the distal (d) ribosome standby domain (222) is 0 to 20 bases in length. In an embodiment, the protein coding sequence (220) encodes for a fluorescent protein, an enzyme, or a growth factor. In an embodiment, the contacting step occurs in a cell-free expression system, in a cell lysate, or in a cell.

The process for regulating protein translation with a toehold exchange (THE) riboregulator (225) involves a series of precisely orchestrated molecular events. The process begins with providing a THE riboregulator gate (225). This gate, comprising an output strand (201) and a gate prime strand (202), is a double-stranded RNA molecule designed to control protein translation. The output strand (201) contains sequences involved in strand exchange, including the input branch migration domain (206), output branch migration domain (204), and output toehold domain (205) which sequesters the ribosome binding sequence (226). The gate prime strand (202) includes sequences for regulating translation, such as the self-cleaving ribozyme sequence (209), gate input toehold domain (210), substrate domain (211), gate prime wobble domain (212), output toehold sequester domain (213), c spacer domain (217), start codon (218), n-terminal linker (219), protein coding sequence (220), toehold spacer domain (221), and transcription termination sequence (214). The next step involves contacting the THE riboregulator gate (225) with an input strand (215), triggering the regulatory process. The input strand (215) comprises an input toehold domain (223), an input branch migration domain (206), and a distal (d) ribosome standby domain (222). The interaction between the input strand (215) and the gate (225) initiates a strand exchange reaction. The input toehold domain (223) hybridizes to the gate input toehold domain (210), promoting branch migration and displacement of the output strand (201). This displacement leads to the formation of a strand exchange product (216), characterized by the input strand (215) hybridized to the gate prime strand (202) and the output strand (201) released. Consequently, the ribosome binding sequence (226) becomes exposed, allowing for translation initiation of the protein coding sequence (220) and production of the translation-regulated protein (224). This controlled activation of protein translation is a key feature of the THE riboregulator system. The provision of the THE riboregulator gate (225) and its subsequent contact with the input strand (215) initiate a precisely controlled series of molecular events, culminating in regulated protein translation. The specific sequences and structural features of the RNA molecules ensure accurate recognition, efficient strand exchange, and precise control over translation initiation. The formation of the strand exchange product (216) marks the transition to the ON state (227), enabling protein production. This orchestrated process provides a robust and versatile mechanism for controlling gene expression.

Providing a THE riboregulator gate (225) can be an initial step in the process of regulating protein translation. The gate (225) is a double-stranded RNA molecule designed to function as a control element for protein expression. It comprises an output strand (201) and a gate prime strand (202). The output strand (201) contains sequences involved in the strand exchange reaction, including a linker (L) sequence (208), an output wobble domain (207), an input branch migration domain (206), an output toehold domain (205), an output branch migration domain (204), and a hairpin-forming sequence (203). The gate prime strand (202) includes sequences essential for regulating translation, namely, a self-cleaving ribozyme sequence (209), a gate input toehold domain (210), a substrate domain (211), a gate prime wobble domain (212), an RBS (226), a c spacer domain (217), a start codon (218), an n-terminal linker (219), a protein coding sequence (220), a toehold spacer domain (221), and a transcription termination sequence (214). One benefit of providing this complex RNA structure is it sets the stage for precise control of protein expression. The gate acts as a molecular switch, with the RBS (226) initially sequestered by the output toehold domain (205), preventing translation. This “OFF” state minimizes leaky expression and establishes a baseline for controlled activation. The gate can be produced in vitro through enzymatic transcription or expressed in vivo within cells, providing flexibility for different applications. Providing a THE riboregulator gate (225) establishes the foundation for controlled and programmable regulation of protein translation. The gate's complex structure, incorporating various functional domains, enables precise control over the strand exchange reaction and subsequent translation initiation. The flexibility in production methods allows adaptation to diverse experimental settings.

Contacting the THE riboregulator gate (225) with an input strand (215) is the triggering event that initiates the regulatory process. The input strand (215) is a single-stranded RNA molecule designed to interact specifically with the gate. It comprises an input toehold domain (223), an input branch migration domain (206), and a distal (d) ribosome standby domain (222). One benefit of this contacting step is initiation of strand exchange. The input toehold domain (223) of the input strand hybridizes to the gate input toehold domain (210) on the gate prime strand (202). This interaction triggers a cascade of molecular events, including branch migration between the input and gate strands, ultimately leading to the displacement of the output strand (201) and the exposure of the ribosome binding sequence (226). The contacting step can occur in vitro, by adding the input strand to a solution containing the gate, or in vivo, by co-expressing both molecules within cells. Variations in the concentrations of the RNA molecules, temperature, and buffer conditions can be used to modulate the kinetics of the reaction. Longer input toeholds generally lead to faster kinetics. Contacting the THE riboregulator gate (225) with the input strand (215) initiates the precisely orchestrated strand exchange reaction that controls protein translation. The sequence specificity of the toehold domains ensures a targeted response to the input signal, while variations in reaction conditions allow for fine-tuning of the system's kinetics and efficiency.

Initiating a strand exchange reaction between the THE riboregulator gate (225) and the input strand (215) is the central event in the regulation of protein translation. The reaction is initiated by the hybridization of the input toehold domain (223) of the input strand to the gate input toehold domain (210) of the gate prime strand (202). One benefit of initiating strand exchange is the controlled displacement of the output strand. This displacement is driven by the branch migration process, where the input branch migration domain (206) progressively hybridizes to the substrate domain (211), replacing the output strand (201). The kinetics of strand exchange can be modulated by adjusting the lengths and sequences of the toehold and branch migration domains, providing fine-tuned control over the regulatory process. Longer input toeholds generally lead to faster kinetics. The initiation of strand exchange marks a critical turning point in the regulation of protein translation. This reaction, driven by toehold binding and branch migration, leads to the controlled displacement of the output strand and sets the stage for the activation of protein synthesis.

Displacing the output strand (201) from the gate prime strand (202) is the key event that activates the THE riboregulator. As the input branch migration domain (206) hybridizes to the substrate domain (211), it progressively displaces the output strand (201), eventually releasing it from the gate prime strand (202). This displacement results in the formation of the strand exchange product (216), which comprises the input strand (215) hybridized to the gate prime strand (202). One benefit of this displacement is exposure of the ribosome binding sequence (226). With the output strand removed, the RBS is no longer sequestered, allowing ribosomes to bind and initiate translation. This transition from the OFF state to the ON state (227) is essential for activating protein production. The stability of the strand exchange product (216) is influenced by the design of the toehold and branch migration domains. The displacement of the output strand (201) and formation of the strand exchange product (216) mark the activation of the THE riboregulator. This event exposes the ribosome binding sequence (226), enabling translation initiation and subsequent protein production.

Initiating translation of the protein coding sequence (220) is the final step in the process, resulting in the production of the desired protein. When the ribosome binding sequence (226) is exposed in the ON state (227) of the THE riboregulator, ribosomes can bind to the RBS and initiate translation of the protein coding sequence (220). One benefit of this translation initiation is the controlled production of the target protein (224). The precise regulation of translation by the THE riboregulator allows researchers to control the timing and level of protein expression in response to specific input signals. This controllability is a valuable tool for manipulating cellular processes and implementing complex regulatory circuits. Variations in translation conditions, such as temperature and magnesium concentration, can be optimized to enhance protein production yields. The choice of protein coding sequence (220) determines the specific protein produced, providing flexibility and customizability to the system. The initiation of translation at the exposed ribosome binding sequence (226) represents the culmination of the THE riboregulator's function, resulting in the controlled production of the translation-regulated protein (224). This precise regulation of protein expression is a powerful tool for manipulating cellular processes and implementing complex genetic circuits.

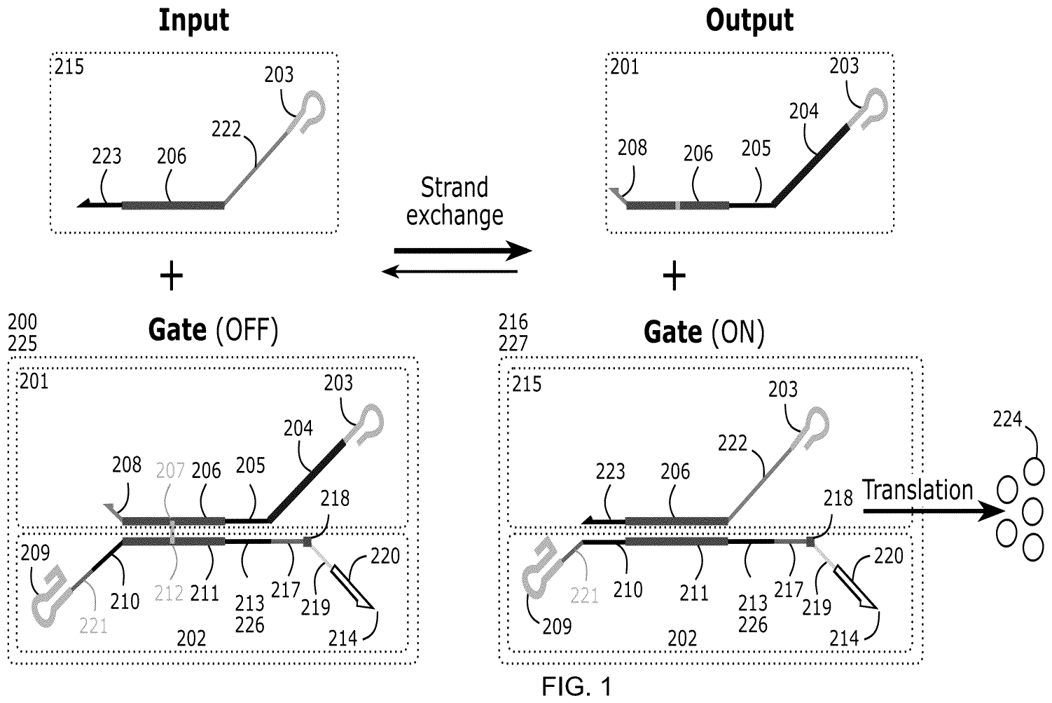

FIG. 1 illustrates the mechanism of toehold exchange (THE) riboregulation and its control over protein translation. The figure depicts the THE riboregulator gate (225) in both its OFF and ON (227) states, highlighting the key components involved in the strand exchange reaction and their roles in regulating protein synthesis. Initially, the THE riboregulator gate (225) is in the OFF state. The output strand (201), represented in blue, is hybridized to the gate prime strand (202), depicted in black. The output toehold domain (205) on the output strand is sequestering the RBS (226) and the gate input toehold domain (210) on the gate prime strand are shown as single-stranded regions, available for interaction with the input strand (215). The input strand (215), shown in red, comprises the input toehold domain (223), the input branch migration domain (206), and the distal ribosome standby domain (222). The input strand initiates the toehold exchange reaction by hybridizing to the gate input toehold domain (210) through its input toehold domain (223). This hybridization initiates branch migration, where the input branch migration domain (206) displaces the output strand (201) by hybridizing to the substrate domain (211) on the gate prime strand. This strand exchange results in the formation of the strand exchange product (216) and the THE riboregulator gate (227) transitioning to the ON state. In the ON state, the output strand (201) is released, and the ribosome binding sequence (226) is exposed. This exposure allows ribosomes to bind to the RBS and initiate translation of the protein coding sequence (220), leading to the production of the translation-regulated protein (224), represented by the purple circles. The distal ribosome standby domain (222) on the input strand can influence the kinetics of the strand exchange reaction and translation initiation rate, providing an additional layer of control over the process. The c spacer domain (217) and the start codon (218) on the gate prime strand are crucial for efficient translation initiation. The figure effectively illustrates the dynamic interplay between the RNA components of the THE riboregulator, the input strand, and the translational machinery, highlighting the mechanism by which this system controls protein expression.

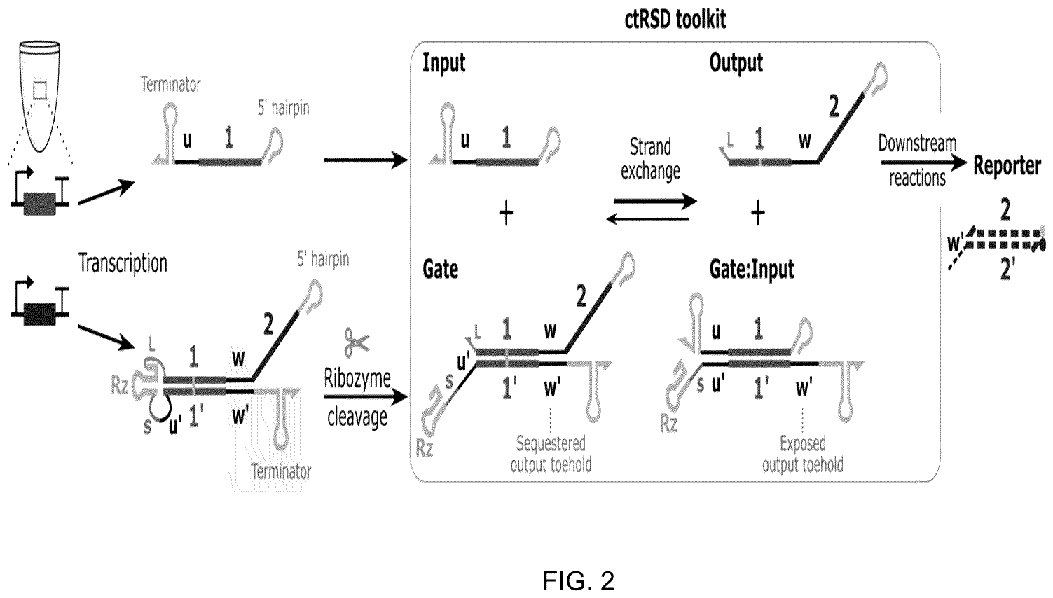

FIG. 2 provides a schematic overview of the process for producing RNA strands from DNA templates and the self-assembly of RNA strands into functional THE riboregulators. An internal self-cleaving ribozyme (Rz) (209) within a transcribed gate hairpin cleaves the transcript, generating a double-stranded RNA (dsRNA) gate suitable for strand exchange with a complementary ssRNA input, releasing an ssRNA output. This process enables the implementation of toehold-mediated strand exchange reactions in RNA-based circuits. The figure effectively illustrates the principles of RNA production, self-assembly of RNA devices, and toehold-mediated strand exchange, providing a modular and programmable platform for constructing RNA-based logic circuits and molecular devices.

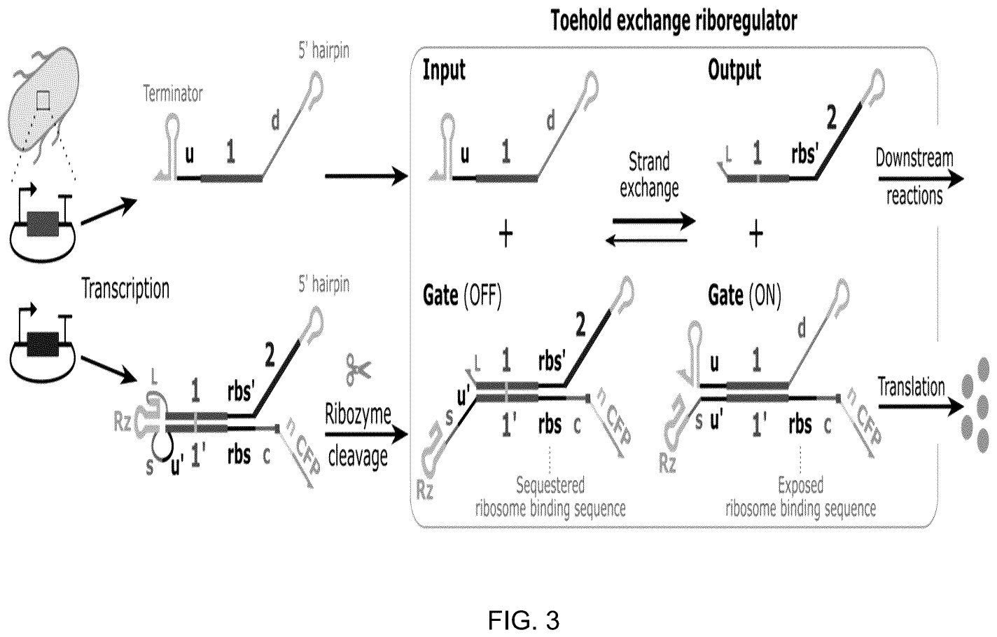

FIG. 3 details the design and operation of a toehold exchange (THE) riboregulator, highlighting its application in controlling protein expression in bacteria. The figure illustrates the mechanism by which RNA strand exchange is coupled to protein production, enabling RNA computation to regulate gene expression and providing a means to measure toehold exchange in bacterial cells. The THE riboregulator functions similarly to the RNA devices described in FIG. 2, but with a crucial modification: the output toehold is replaced with a ribosome binding sequence (RBS) (226). After transcription and ribozyme cleavage, the THE riboregulator is initially in the OFF state (225). The RBS (226) is sequestered, preventing translation of the downstream CDS (220). However, when a complementary input strand (215) is present, strand exchange occurs. The input strand binds to the gate input toehold domain (210), initiating branch migration and displacing the output strand (201). This displacement exposes the RBS, enabling ribosome binding and initiating translation of the CDS. The newly synthesized protein, in this example CFP, can then be measured as a readout of riboregulator activity. This process is termed “toehold exchange” because the base pairing of the input toehold is exchanged for the dissociation of the ribosome binding sequence (RBS) sequestering domain (228). THE riboregulators can be tested by cloning the input and riboregulator gates onto separate plasmids, transforming these plasmids into E. coli cells, and measuring cellular fluorescence using flow cytometry. The figure effectively illustrates how RNA strand exchange can be coupled to protein production, creating a genetically encodable system for controlling gene expression in bacteria.

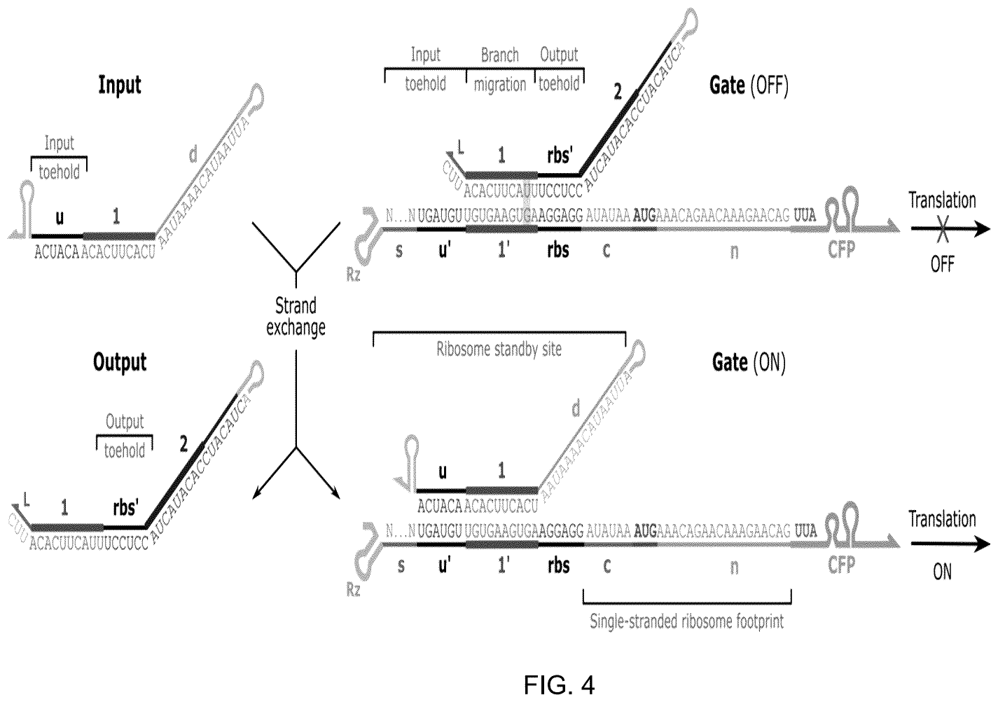

FIG. 4 presents a detailed sequence schematic of a toehold exchange (THE) riboregulator, illustrating the RNA sequences involved and their interactions in both the OFF and ON (227) states. The figure provides a deeper understanding of the sequence-level design principles underlying THE riboregulation and highlights the importance of specific sequence elements in controlling translation. The top left portion of the figure shows a schematic of the input strand (215). It comprises, from 5′ to 3′, the distal (d) ribosome standby domain (222), the input branch migration domain, labeled ‘1’, and the input toehold domain (223), labeled ‘u’. The input toehold domain (223) is designed to be complementary to the gate input toehold domain (210) on the THE riboregulator gate, while the input branch migration domain is complementary to the substrate domain (211). The distal ribosome standby domain (222) can modulate the kinetics of strand exchange and the translation initiation rate. The bottom part of the figure illustrates the THE riboregulator gate in the OFF state (225). The gate prime strand (202) includes the self-cleaving ribozyme sequence (Rz) (209), followed by the gate input toehold domain (210), the substrate domain (211) labeled as ‘s’, the RBS (226), the c spacer domain (217), the start codon (218) usually AUG, the n-terminal linker (219) labeled ‘n’, and the protein coding sequence (CDS) (220) encoding CFP. The output strand (201) includes the hairpin forming sequence (203), the output branch migration domain, the output toehold domain (205) labeled as ‘rbs’, the input branch migration domain complementary to the substrate domain (211) on the gate prime strand, the output wobble domain, and the linker (L) sequence (208). In the OFF state, the ribosome binding sequence is sequestered by the output toehold domain (205), preventing translation. The top right and bottom right portions of FIG. 4 depict the output strand (201) and the THE riboregulator gate (227) in the ON state, respectively. In the ON state, the input strand has displaced the output strand through branch migration, exposing the ribosome binding sequence (rbs/226) and enabling translation of the CDS (220). The figure clarifies that the ‘s’ domain can be extended as a spacer or, with extensions of the input strand's 3′ end, can form a longer toehold for enhanced strand exchange initiation. This design feature, denoted as ‘Ns,’ provides flexibility in tuning the kinetics of the reaction. The single-stranded ribosome footprint in the ON state emphasizes the accessibility of the RBS for translation initiation.

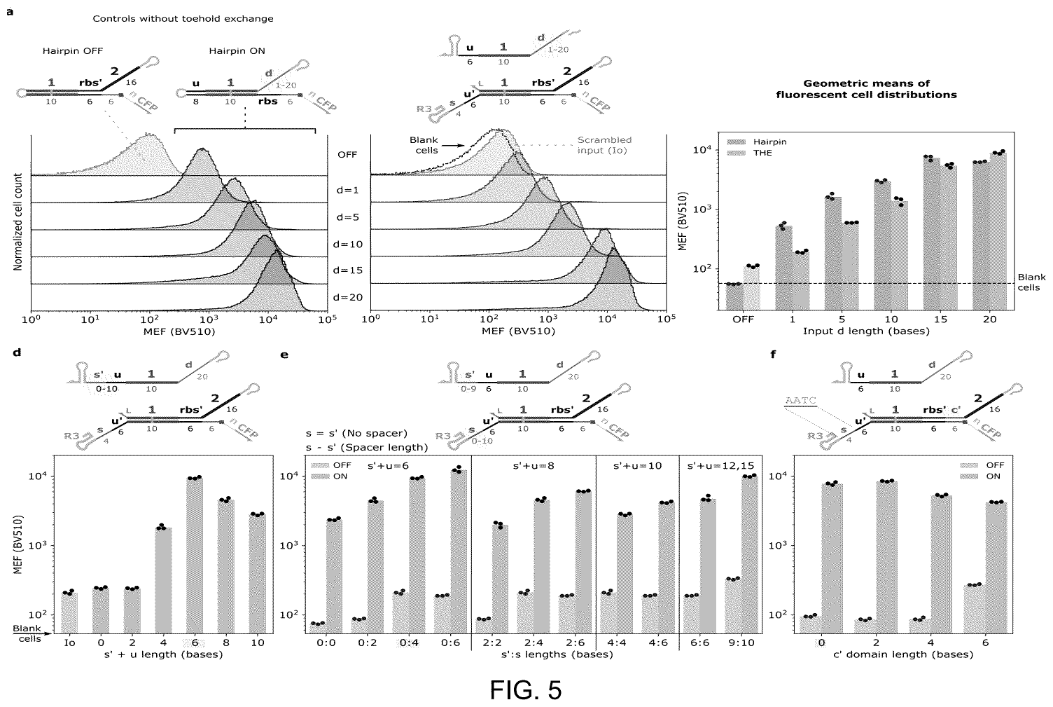

FIG. 5 presents a comprehensive characterization of the toehold exchange (THE) riboregulator design space in E. coli, exploring the effects of varying different domain lengths on protein expression levels. The figure includes schematics of the constructs used, along with plots and bar graphs depicting the results of flow cytometry experiments. The yellow highlighted domains in the schematics above the plots represent the domains varied in the accompanying panels, facilitating direct comparison and analysis of the effects of these variations. Molecules of equivalent fluorophore (MEF) are used as a measure of fluorescence intensity, providing a quantitative readout of protein expression. Results labeled ‘OFF’ represent gates co-transcribed with a non-complementary input, serving as a baseline for comparison. Panels (a) and (b) investigate the translation strength of THE riboregulator designs with different distal (d) ribosome standby domain (222) lengths. Panel (a) focuses on hairpin control constructs that do not require strand exchange for activation, while panel (b) examines separate input and gate constructs where strand exchange is necessary for gate activation. The plots in these panels display the distribution of fluorescence intensities across cell populations, providing insights into the heterogeneity of protein expression. Panel (c) presents bar plots of the geometric means of the cell distributions shown in panels (a) and (b). These bar plots summarize the overall effect of varying ‘d’ domain length on protein expression levels, allowing for direct comparison between the hairpin control and toehold exchange constructs. Panels (d), (e), and (f) further explore the THE riboregulator design space by characterizing the effects of different input toehold lengths (panel d), input toehold and spacer lengths (panel e), and output toehold lengths (panel f), respectively. The samples highlighted in yellow on the x-axes in these panels indicate the ‘canonical’ designs used in the rest of the study, providing a reference point for comparison. The experimental procedures involved cloning the inputs and gates onto separate plasmids with compatible origins of replication, transforming these plasmids into BL21 Star (DE3) E. coli cells, and culturing the cells with IPTG to induce T7 RNA polymerase expression. Flow cytometry was then used to measure cellular fluorescence, providing quantitative data on protein expression levels. Replicates represent three independent colonies picked after transformation, demonstrating the reproducibility of the results. Generally, longer toeholds lead to faster kinetics. The comprehensive analysis presented in FIG. 5 provides valuable insights into the design principles and tunability of THE riboregulators, establishing a foundation for their application in controlling gene expression.

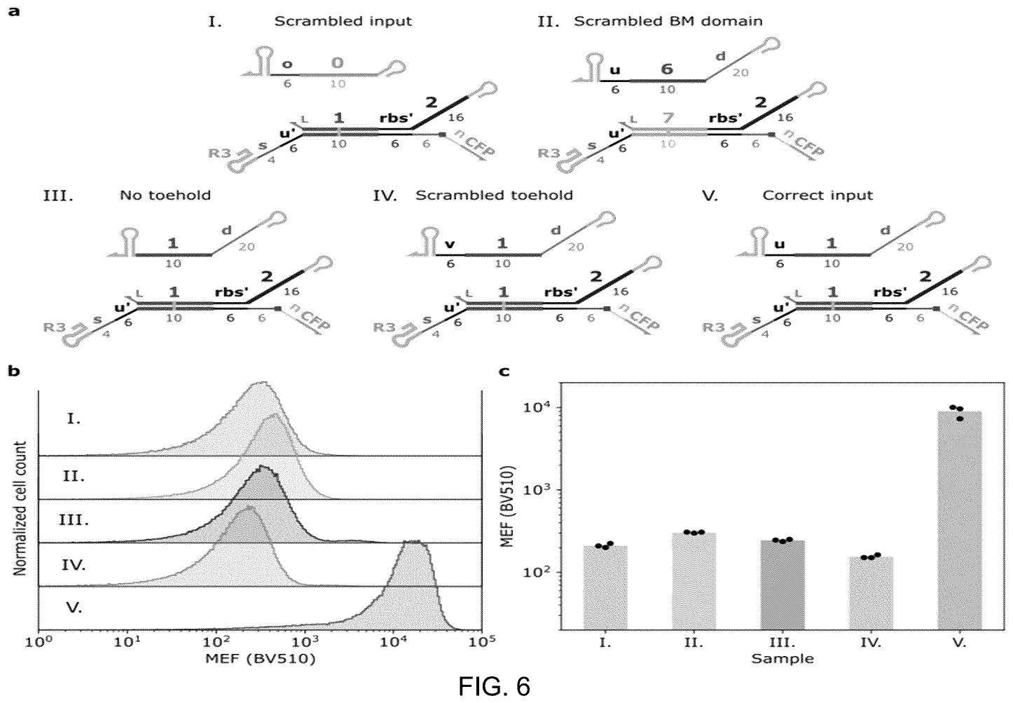

FIG. 6 demonstrates the sequence specificity of toehold exchange riboregulators, highlighting the importance of correct toehold and branch migration domain sequences for achieving robust protein expression. The figure includes schematics of the constructs used and presents flow cytometry data illustrating the results of the experiments. Panel (a) depicts five different constructs used to assess sequence specificity. In sample I, both the toehold and the branch migration domain of the input are not complementary to the gate. This serves as a negative control, demonstrating the lack of protein expression when there is no sequence complementarity. In sample II, the input toehold is complementary to the gate, but the branch migration domain is not. This construct tests the importance of the branch migration domain for successful strand exchange and subsequent protein production. In samples III and IV, the input toehold is either missing or not complementary to the gate, respectively. Sample III replicates the ‘no toehold’ condition from FIG. 5d, providing a direct comparison and further emphasizing the necessity of the toehold for riboregulator activation. In sample V, both the toehold and branch migration domains of the input are fully complementary to the gate. This serves as the positive control, demonstrating maximal protein expression under ideal conditions. Panel (b) presents the fluorescent cell distributions obtained from flow cytometry for each of the constructs depicted in panel (a). The x-axis represents fluorescence intensity (MEF), providing a quantitative measure of protein expression levels. The y-axis represents the normalized cell count, indicating the proportion of cells exhibiting a particular fluorescence intensity. The gray shaded curve represents the negative control (sample I), showing minimal fluorescence. The curves for samples II, III, and IV exhibit increasing levels of fluorescence, reflecting the varying degrees of sequence complementarity and their influence on riboregulator activation. The red curve represents the positive control (sample V), demonstrating the highest fluorescence intensity. Panel (c) shows the geometric means of the fluorescent cell distributions for the constructs. This summarizes the overall effect of each construct on protein expression levels, facilitating direct comparison between the samples. The bar plot clearly shows that substantial protein expression is observed only when both the toehold and branch migration domains of the input are complementary to the gate, confirming the sequence specificity of the THE riboregulators. The experimental setup involved cloning the input and riboregulator gates onto separate plasmids, transforming them into E. coli cells, and inducing protein expression with IPTG. Flow cytometry was used to measure cellular fluorescence. Generally, longer toeholds lead to faster kinetics.

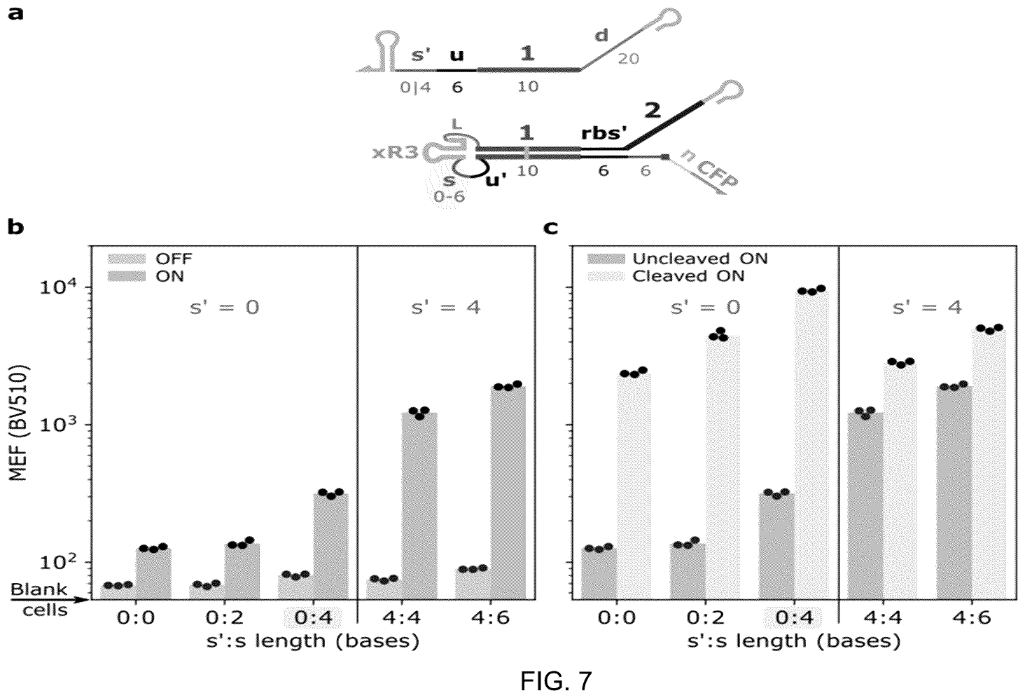

FIG. 7 clarifies that the self-cleaving ribozyme sequence (209) produces a dsRNA gate suitable for strand exchange. Without the self-cleaving ribozyme sequence (209) the gate would be a hairpin structure and strand exchange would not occur, which would lead to significantly lower levels of protein production. Panel (a) shows the schematic of the uncleaved ribozyme constructs tested. The ‘xR3’ construct has a single mutation relative to the functional ‘R3’ ribozyme that abolishes its cleavage activity. The length of the spacer (‘s’) domain (211) within the gate was varied and tested with inputs having either 6 bases (s′=0) or 10 bases (s′=4) of complementarity with the gate toehold. This design allows for the examination of the interplay between spacer length and input toehold complementarity in the absence of ribozyme cleavage. Panel (b) presents the characterization of the uncleaved constructs with complementary (ON) or non-complementary (OFF) inputs. The bar plots display the mean equivalent fluorophore (MEF) values (BvS10) of E. coli cell populations, representing protein expression levels. The gray bars represent the OFF state, while the pink bars represent the ON state. The ‘Blank cells’ bar serves as a negative control, indicating background fluorescence in cells without any fluorescent protein. As expected, the OFF state exhibits lower fluorescence than the ON state across all spacer lengths and input toehold complementarities. Panel (c) compares the uncleaved versus cleaved riboregulator gates with complementary inputs. The light pink bars represent the uncleaved gates, while the darker pink bars represent the cleaved gates. This direct comparison highlights the impact of ribozyme cleavage on protein expression levels. The samples highlighted in yellow on the x-axes in panels (b) and (c) represent the ‘canonical’ designs, providing a reference for comparison. The results reveal that when s′=0 (6-base input toehold), there is significantly lower protein production for the uncleaved gates compared to the cleaved gates. Increasing the spacer length and the complementarity of the input and gate toeholds (s′=4, 10-base input toehold) increases protein production for the uncleaved gates, likely due to loop-mediated invasion of the hairpin structure. Generally, longer toeholds lead to faster kinetics. This observation suggests that longer toeholds can partially compensate for the lack of ribozyme cleavage by promoting strand invasion, albeit with lower efficiency compared to the cleaved gates. The experimental procedures involved cloning the constructs onto separate plasmids, transforming them into E. coli cells, inducing protein expression, and measuring cellular fluorescence using flow cytometry. The findings presented in FIG. 7 demonstrate that ribozyme cleavage is necessary for high protein expression with the canonical THE riboregulator design and highlight the interplay between ribozyme activity, toehold length, and spacer length in modulating gene expression.

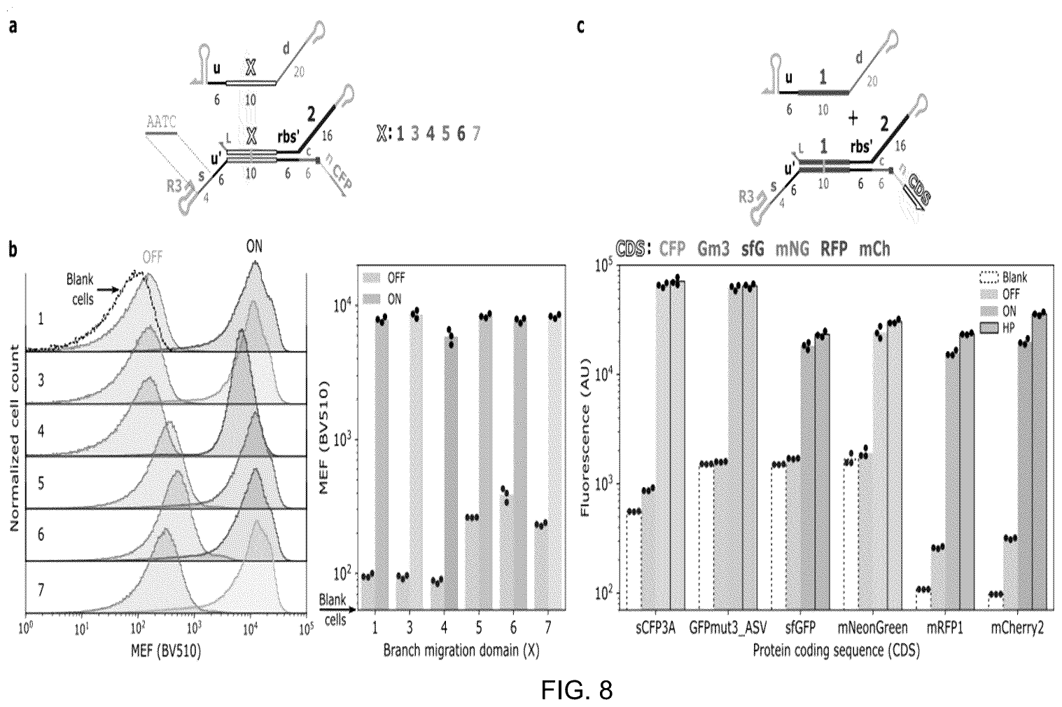

FIG. 8 demonstrates the programmability and modularity of THE riboregulators, highlighting their ability to be customized for different input and output sequences. The figure comprises schematics of the riboregulator gates, flow cytometry plots depicting fluorescent cell distributions, and bar graphs summarizing protein expression levels. Panel (a) shows a schematic of THE riboregulator gates with different input branch migration (BM) domains (206). The branch migration sequences (1, 3, 4, 5, 6, 7) were taken from a previously characterized in vitro ctRSD toolkit and directly implemented in THE riboregulators. This demonstrates the modularity of the system, as different BM domains can be readily swapped without affecting the overall riboregulator design. The ‘x’ in the schematic represents the variable BM domain. Panel (b) presents the results of characterizing these different BM domains in E. coli. The left side shows fluorescent cell distributions obtained from flow cytometry. The x-axis represents fluorescence intensity (MEF, BvS10), while the y-axis represents the normalized cell count. Each curve corresponds to a different BM domain. The gray shaded curves represent the ‘OFF’ state, where the gates were co-transcribed with a non-complementary input. The colored curves represent the ‘ON’ state, with each color corresponding to a specific BM domain. The right side of panel (b) displays bar plots summarizing the geometric means of the cell distributions for the different BM domains. This provides a quantitative measure of protein expression levels for each BM domain in both the OFF and ON states. Panel (c) further demonstrates the modularity of THE riboregulators by characterizing gates regulating the expression of different protein coding sequences (CDSs) (220). The bar graph displays fluorescence intensities (AU) for different CDSs, including CFP, GFP, mNeonGreen, mRFP1, and mCherry2. ‘Blank’ indicates cells without any fluorescent protein at the relevant excitation and emission wavelengths, serving as a negative control. ‘HP’ represents cells with a constitutively ON hairpin construct, as shown in FIG. 5a, providing a reference for maximal expression levels. The results in panel (c) demonstrate that THE riboregulators can effectively control the expression of various fluorescent proteins, showcasing the system's adaptability and broad applicability. The experimental procedures involved cloning the constructs into plasmids, transforming them into E. coli cells, inducing protein expression, and measuring fluorescence using flow cytometry. The use of different BM domains and CDSs highlights the modularity and programmability of THE riboregulators for controlling gene expression. Generally, longer toeholds lead to faster kinetics.

FIG. 9 explores the functionality of orthogonal ribozymes and toeholds from the ctRSD toolkit within THE riboregulators in E. coli cells. The figure uses schematics of the riboregulators and bar graphs depicting geometric means of fluorescent cell distributions to illustrate the effects of different ribozyme and toehold combinations. Panels (a) and (b) depict schematics of THE riboregulators with different HDV-like ribozymes and spacer sequences, combined with either the ‘u’ input toehold or the ‘v’ input toehold, respectively. These toeholds and ribozyme sequences were previously characterized in vitro as part of the ctRSD toolkit. The spacer (‘s’) domains (211) were designed with slightly different sequences depending on the ribozyme and input toehold combination to minimize undesired secondary structure formation, as predicted by NUPACK. Spacer domains ‘4u’ and ‘4v’ indicate spacers designed to have additional complementarity with the ‘s” domains on the inputs. All ‘4s’ domains are designed with a C base adjacent to the toehold to prevent unintended base pairing with inputs possessing longer toeholds or outputs from upstream gates. This careful design of spacer sequences ensures that the riboregulators fold correctly and function as intended. Panel (c) presents the results of the experiments, showing the geometric means of fluorescent E. coli distributions from flow cytometry for the constructs shown in panels (a) and (b). The x-axis labels indicate the ribozyme and spacer combinations used. ‘R3’ represents the canonical ribozyme used in previous figures. ‘Rh’, ‘Rg’, and ‘Rm’ represent alternative ribozymes from the ctRSD toolkit, while ‘4s’, ‘45w’, ‘45a’, ‘4u’, and ‘4v’ indicate different spacer sequences. The gray bars represent the OFF state, where the gates were co-transcribed with a non-complementary input, providing a baseline for comparison. The pink bars represent the ON state, with complementary inputs, indicating riboregulator activation. The ‘Blank cells’ bar represents the background fluorescence of cells without any fluorescent protein. The results demonstrate that orthogonal ribozymes (Rh, Rg, Rm) and toeholds (u, v) from the ctRSD toolkit function effectively in THE riboregulators in cells. The observed fluorescence levels in the ON state indicate successful strand exchange and protein production, confirming the compatibility and functionality of these orthogonal components. The experimental procedures involved cloning the constructs into plasmids, transforming them into E. coli cells, inducing protein expression, and measuring cellular fluorescence using flow cytometry. The use of orthogonal ribozymes and toeholds expands the design space and potential applications of THE riboregulators, allowing for the construction of more complex and independent control circuits. Generally, longer toeholds lead to faster kinetics.

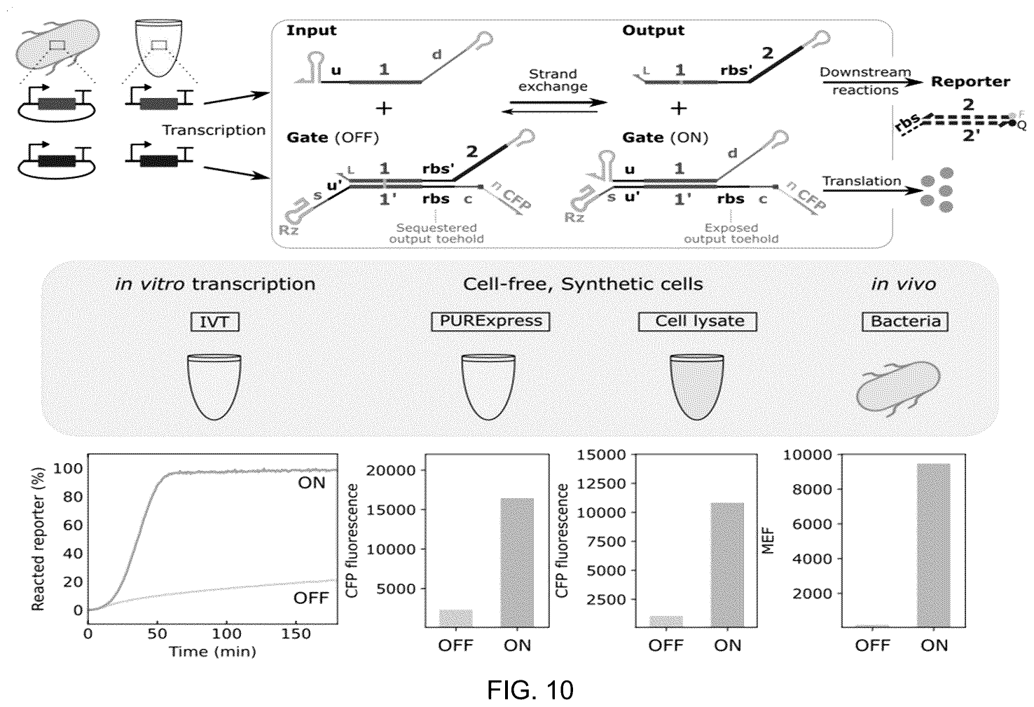

FIG. 10 demonstrates the versatility of toehold exchange riboregulators (using co-transcriptional RNA strand displacement (ctRSD) circuits) showcasing their ability to operate across different environments, from simple in vitro transcription reactions to in vivo bacterial cells. The figure comprises a schematic of a THE riboregulator circuit and presents experimental data illustrating circuit performance in various settings. The top panel shows a schematic of a THE riboregulator, illustrating the input (215), gate (225), and output (224) components. The input strand (215) interacts with the gate (225), leading to strand exchange and exposure of the ribosome binding sequence (226). This exposure triggers translation of the protein coding sequence, resulting in the production of the output protein (224). The ribozyme (Rz) plays a crucial role in the co-transcriptional formation of the gate, producing a dsRNA gate suitable for strand exchange. The bottom panel presents experimental data demonstrating the functionality of toehold exchange riboregulators in four different environments: simple in vitro transcription reactions (IVT), PURExpress cell-free transcription-translation reactions, BL21 Star (DE3) bacterial cell lysate, and BL21 Star (DE3) cells. The IVT reactions demonstrate the basic functionality of strand exchange and output production. The graph shows the percentage of reacted reporter over time, with the ‘ON’ state (blue curve) exhibiting significantly higher reporter activity than the ‘OFF’ state (gray curve). The PURExpress reactions showcase the coupling of strand exchange to protein production in a cell-free environment. The bar graph displays CFP fluorescence for both the ‘OFF’ and ‘ON’ states, with the ‘ON’ state exhibiting higher fluorescence, indicating successful protein production. The bacterial cell lysate experiments demonstrate the functionality of the circuits in a complex biological environment containing cellular components. Similar to the PURExpress reactions, the bar graph shows CFP fluorescence for both states, with the ‘ON’ state exhibiting higher fluorescence. Finally, the experiments in BL21 Star (DE3) cells validate the in vivo functionality of the circuits in living bacterial cells. The bar graph depicts MEF values, a measure of cellular fluorescence, with the ‘ON’ state showing higher fluorescence than the ‘OFF’ state, confirming successful protein expression in vivo. The figure effectively demonstrates the robustness and adaptability of toehold exchange riboregulators across diverse environments, highlighting their potential for various synthetic biology applications. Generally, longer toeholds lead to faster kinetics.

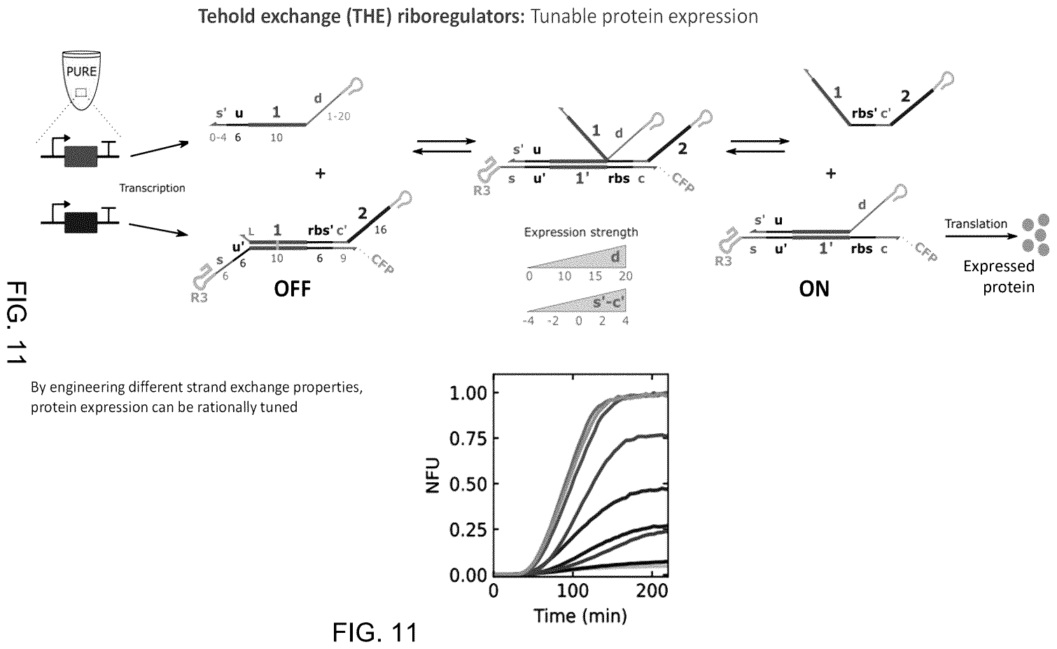

FIG. 11 provides a schematic illustration of toehold exchange (THE) riboregulators and their function in a cell-free PURE system, emphasizing the tunability of protein expression through engineering different strand exchange properties. Longer distal domains (222) lead to slower activation. The figure depicts the THE riboregulator in both its OFF (225) and ON (227) states, and it includes a graph illustrating the tunable expression strength achieved by varying the length of the distal (d) ribosome standby domain (222). The top left of the figure shows a representation of a PURE cell-free expression system. A double-stranded DNA template (red and blue lines) encoding the THE riboregulator is transcribed by T7 RNA polymerase (gray oval) to produce the RNA riboregulator (blue line). The ‘T’ represents the terminator sequence. The schematic to the right of the PURE system illustration shows the THE riboregulator in its OFF state (225). The R3 ribozyme sequence (209) is indicated, along with the ‘s’, ‘u’, ‘1’, ‘rbs”, ‘c’, and ‘CFP’ domains, representing the substrate domain (211), input toehold (210), branch migration domain, sequestered ribosome binding sequence (213/226), c spacer domain (217), and the cyan fluorescent protein (CFP) coding sequence (220), respectively. The input RNA strand (215) is shown separately with its ‘s”, ‘u’, ‘1’, and ‘d’ domains. The central part of the figure illustrates the strand exchange process. The input strand (215) interacts with the THE riboregulator gate (225) through toehold-mediated branch migration, leading to the displacement of the output strand and exposure of the ribosome binding sequence. The schematic on the right depicts the THE riboregulator in the ON state (227). The ribosome binding sequence is now accessible, enabling translation of the CFP coding sequence and resulting in the expression of the CFP protein, indicated by the cyan circles. The bottom of FIG. 11 includes a graph showing the time course of CFP expression (NFU) for THE riboregulators with varying ‘d’ domain lengths. The x-axis represents time in minutes, while the y-axis represents normalized fluorescence units (NFU). Each curve corresponds to a different ‘d’ domain length (1, 5, 10, 15, 20), with longer ‘d’ domains resulting in slower activation kinetics. This demonstrates how protein expression strength can be rationally tuned by engineering different strand exchange properties. Generally, longer toeholds lead to faster kinetics.

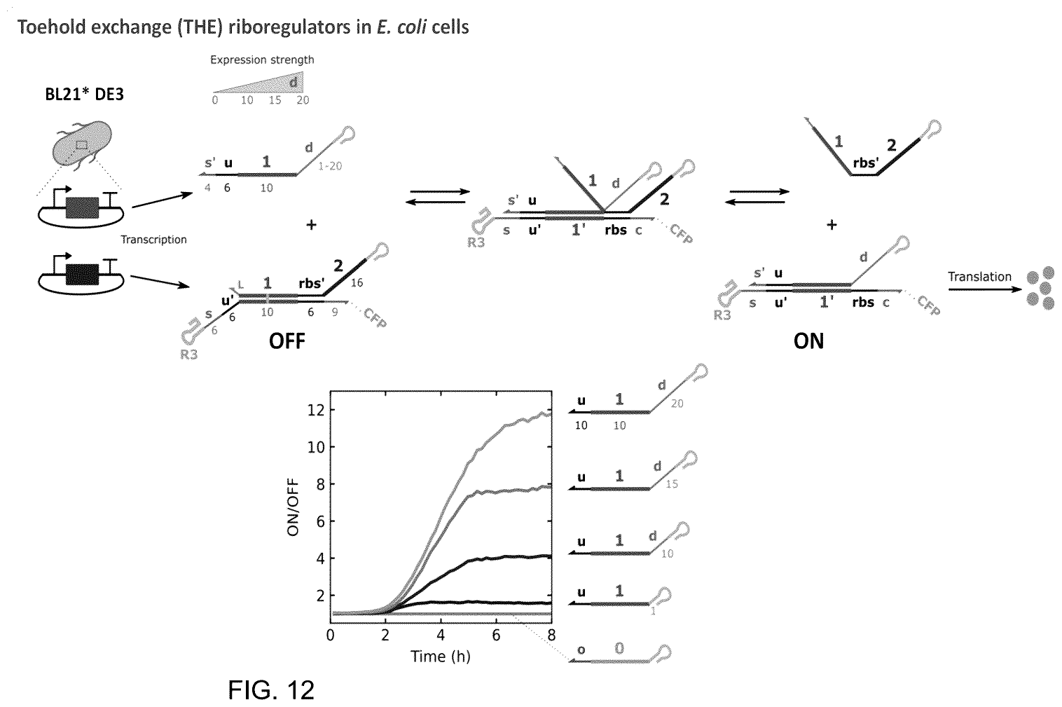

FIG. 12 extends the characterization of THE riboregulators to E. coli cells, demonstrating their functionality in a cellular environment and highlighting the tunability of protein expression strength. The figure includes schematics of the riboregulator constructs, along with a graph illustrating the time course of protein expression for varying distal (d) ribosome standby domain (222) lengths. Longer distal domains (222) lead to slower activation. The top left portion of the figure depicts the BL21 DE3 E. coli expression system. A plasmid (red circle) containing the DNA sequence encoding the THE riboregulator, along with a separate input plasmid, is introduced into the bacterial cell. Transcription of the DNA template produces the RNA riboregulator (blue line), which interacts with the input RNA (red line). The ‘T’ represents the terminator sequence. Adjacent to this illustration, a schematic depicts the THE riboregulator in the OFF state (225). The R3 ribozyme (209), ‘s’, ‘u’, ‘1’, ‘rbs”, ‘c’, and ‘CFP’ domains are indicated, representing the ribozyme sequence, substrate domain (211), input toehold (210), branch migration domain, ribosome binding sequence (213/226), c spacer (217), and CFP coding sequence (220), respectively. The input strand (215), shown with ‘s”, ‘u’, ‘1’, and ‘d’ domains, interacts with the gate, initiating strand exchange. The central part of FIG. 12 illustrates the strand exchange reaction. The input strand (215) binds to the gate through toehold-mediated branch migration, displacing the output strand and exposing the ribosome binding sequence. The schematic to the right shows the THE riboregulator in the ON state (227), with the RBS accessible for translation initiation. The CFP protein, represented by the cyan circles, is produced. The bottom part of the figure presents a graph illustrating the time-dependent expression of CFP in E. coli cells for THE riboregulators with varying ‘d’ domain lengths on the input strand. The x-axis represents time in hours, while the y-axis represents the ON/OFF ratio of fluorescence, providing a normalized measure of riboregulator activity. Each curve corresponds to a different ‘d’ domain length (10, 15, 20), with shorter ‘d’ domains generally resulting in faster activation kinetics and higher expression levels. The graph demonstrates that protein expression strength can be rationally tuned by varying the length of the ‘d’ domain, influencing the kinetics of strand exchange. The experimental setup involved cloning the constructs onto separate plasmids, transforming them into E. coli cells, and measuring fluorescence over time. Generally, longer toeholds lead to faster kinetics.

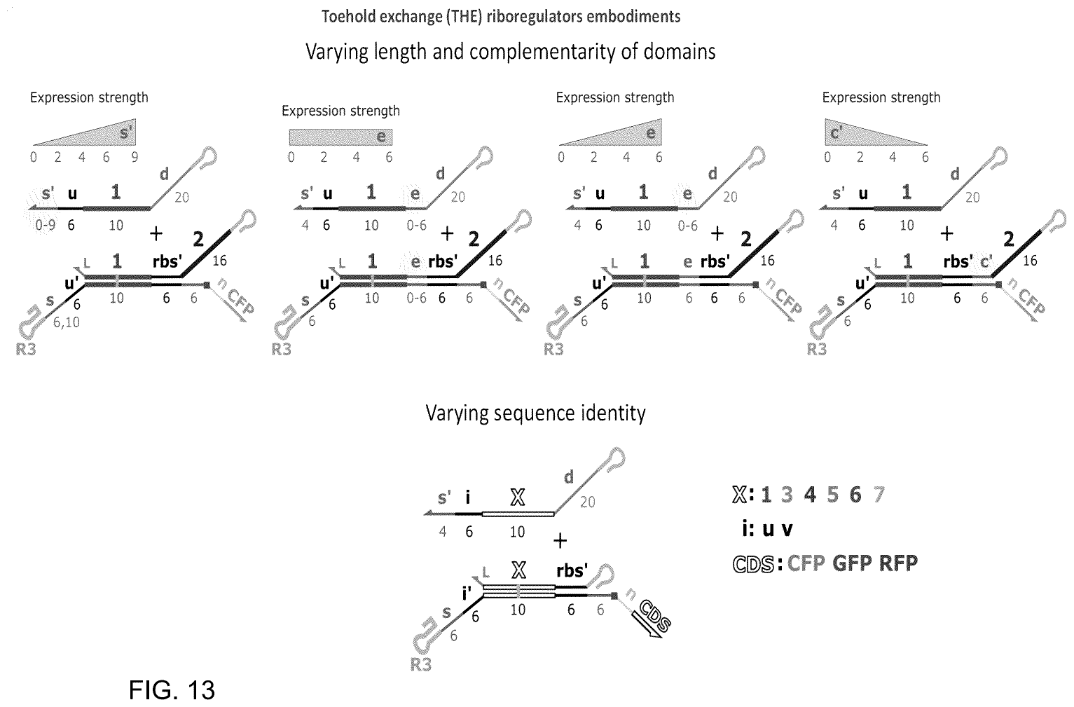

FIG. 13 further explores the design variations of toehold exchange (THE) riboregulators, focusing on the impact of varying the length and complementarity of different domains on expression strength. Generally, longer toeholds lead to faster kinetics, but shorter distal (d) domains (222) also lead to faster kinetics and higher expression. The figure presents schematic representations of several riboregulator embodiments, highlighting the variable domains and their potential impact on the system's performance. The top part of the figure depicts four different riboregulator embodiments, each with a distinct variation in domain length or complementarity. The expression strength, a qualitative measure of protein production, is indicated below each schematic, ranging from low to high. The first schematic shows a riboregulator with a variable ‘s” domain length, ranging from 0 to 6 bases. This domain, located on the input strand (215), corresponds to the region complementary to the spacer domain on the gate prime strand (202). Variations in the ‘s” domain length affect the toehold length and thus influence the kinetics of strand exchange. The second schematic depicts a riboregulator with a variable ‘e’ domain length, ranging from 0 to 6 bases. This ‘e’ domain represents an extension of the output toehold domain (205), which influences the stability of the output strand (201) and its interaction with the ribosome binding sequence (226). The third schematic illustrates a riboregulator with a variable ‘c’ domain length, ranging from 0 to 4 bases. This ‘c’ domain corresponds to the spacer region (217) between the RBS (226) and the start codon (218). Variations in ‘c’ domain length affect the spacing between the ribosome binding site and the start codon, influencing translation initiation efficiency. The fourth schematic shows a riboregulator with a variable ‘c” domain length, ranging from 0 to 4 bases, representing another spacer region on the input strand. The bottom part of FIG. 13 depicts two additional riboregulator designs, exploring variations in sequence identity. The branch migration (X) and input toehold domains can be varied, with ‘X’ representing different branch migration sequences (1, 3, 4, 5, 6, 7) from the ctRSD toolkit, and ‘i’ representing different input toeholds (‘u’ or ‘v’). Further, the coding sequence can be varied, with ‘CDS’ representing different fluorescent proteins (GFP or RFP). These variations highlight the modularity of the THE riboregulator system. The R3 ribozyme (209) is indicated in the schematics, along with other key domains such as ‘s’, ‘u’, ‘1’, ‘rbs’, and ‘CFP’.

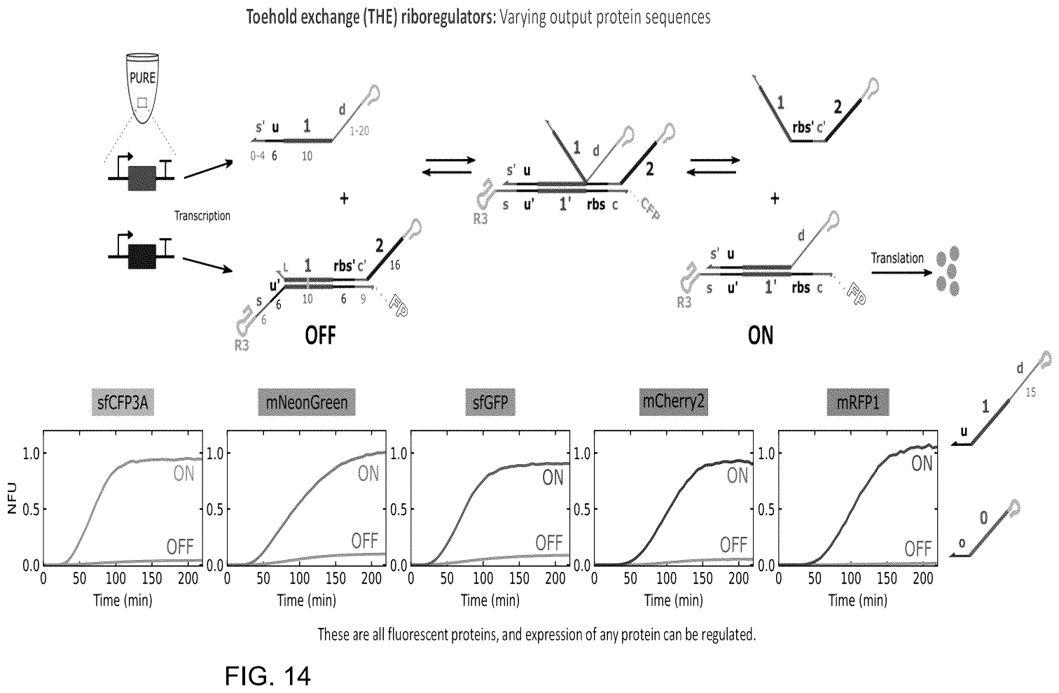

FIG. 14 demonstrates the versatility of THE riboregulators in controlling the expression of various output protein sequences, highlighting the system's modularity and adaptability for different applications. The figure includes schematics of the riboregulator constructs and presents graphs illustrating the time course of fluorescent protein expression for different coding sequences. The top part of FIG. 14 shows a schematic representation of THE riboregulators with varying output protein sequences. The left side depicts the riboregulator in the OFF state (225). The R3 ribozyme (209), ‘s’, ‘u’, ‘1’, ‘rbs' (226), ‘c’ (217), and ‘CDS’ domains are indicated, representing the ribozyme sequence, substrate domain (211), input toehold (210), branch migration domain, ribosome binding sequence, c spacer, and coding sequence (220), respectively. The input RNA (red) interacts with the gate, initiating strand exchange. The central part of the schematic illustrates the strand exchange process. The input strand binds to the gate through toehold-mediated branch migration, displacing the output strand and exposing the RBS. The schematic on the right depicts the riboregulator in the ON state (227), with the RBS accessible and translation of the CDS occurring. The expressed protein is represented generically by a gray circle. The variable ‘s” and ‘d’ domains on the input strand (215), ranging from 0-4 and 1-20 bases, respectively, allow for tuning of the strand exchange kinetics and translation initiation rate. Longer distal (d) domains lead to slower activation. The bottom part of FIG. 14 presents graphs illustrating the time course of fluorescent protein expression for five different coding sequences: sfCFP3A, mNeonGreen, sfGFP, mCherry2, and mRFP1. The x-axis represents time in minutes, while the y-axis represents normalized fluorescence units (NFU), providing a quantitative measure of protein expression. Each graph includes two curves: the gray curve represents the OFF state (with a non-complementary input), while the colored curve represents the ON state (with a complementary input). The ON state curves show a significant increase in fluorescence over time, indicating successful protein production. The figure emphasizes that these are all fluorescent proteins, and that the expression of any protein can be regulated using this system. The experimental setup involved cloning the riboregulator constructs with different CDSs into plasmids, transforming them into E. coli or other appropriate host cells, inducing protein expression, and measuring fluorescence over time. Generally, longer toeholds lead to faster kinetics.

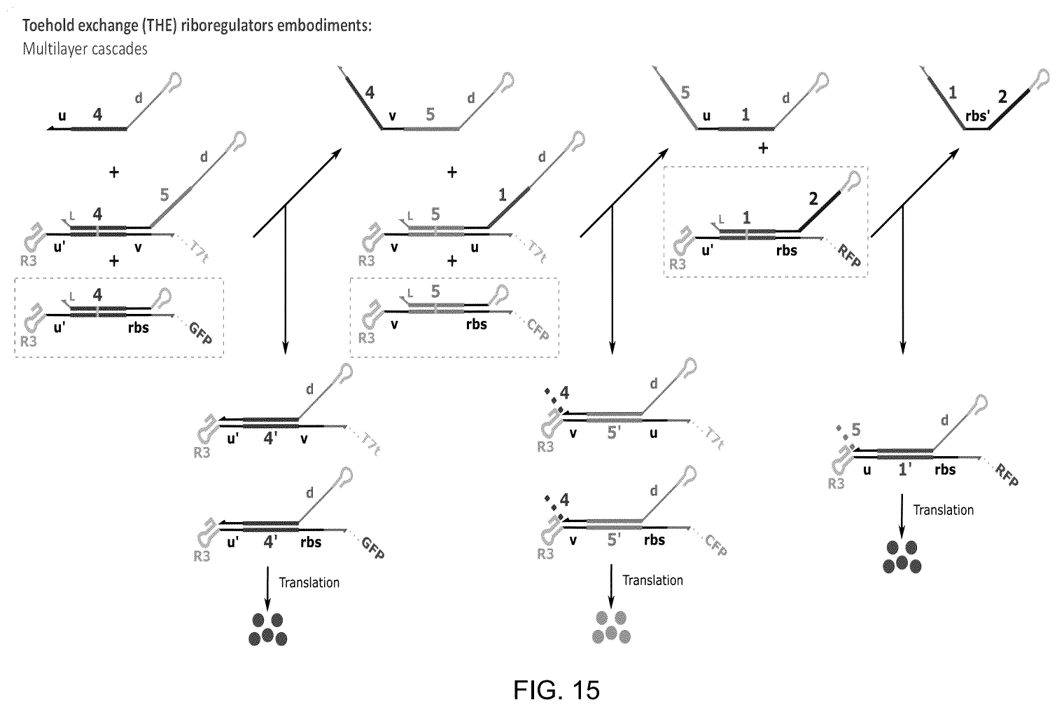

FIG. 15 illustrates the implementation of multilayer cascades using THE riboregulators, demonstrating the potential for building complex logic circuits and achieving sophisticated control over gene expression. The figure presents schematics of a three-layer cascade, highlighting the interactions between different riboregulator gates and their respective input and output strands. The top row of FIG. 15 depicts the individual components of the cascade. The leftmost schematic shows a THE riboregulator gate with an input toehold ‘u’ and an output ‘4’. The central schematic shows another THE riboregulator gate with an input toehold ‘v’ and an output ‘5’. The rightmost schematic depicts a THE riboregulator gate with an input toehold ‘u’ and an output ‘1’, coupled to an RFP coding sequence. The ‘d’ domains on the output strands represent distal ribosome standby domains that can influence translation initiation rate. The second row illustrates the first layer of the cascade. The output ‘4’ from the first gate serves as the input for a subsequent layer. The ‘u’ input and ‘v’ input trigger their respective gates, initiating strand exchange. The third row represents the second layer of the cascade. The output ‘5’ from the second gate serves as an input for the final layer. The output ‘1’ from the third gate triggers the expression of RFP. The bottom row of FIG. 15 shows the final output of the cascade. The expression of RFP is represented by the red circles. The ‘T7t’ denotes the transcription terminator sequence. The R3 ribozyme is indicated in each gate, producing a dsRNA gate suitable for strand exchange. The ‘s’, ‘u’, and ‘v’ domains represent the substrate domain, input toehold ‘u’, and input toehold ‘v’, respectively. The ‘rbs' domain indicates the ribosome binding sequence. The cascade functions as follows: The input ‘u’ activates the first gate, producing the output ‘4’. The output ‘4’ then serves as the input ‘v’ for the second gate. The input ‘v’ activates the second gate, generating the output ‘5’. This output ‘5’ serves as the input ‘u’ for the third gate. Finally, activation of the third gate leads to the expression of RFP. This multilayer cascade demonstrates how THE riboregulators can be linked together to create complex logic circuits and control gene expression in a programmable manner. Generally, longer toeholds lead to faster kinetics.

Traditional riboregulators, often based on RNA hairpins, rely on unimolecular conformational changes for regulatory function. These systems can be limited in their dynamic range and modularity, posing challenges for precise control of gene expression and integration into complex genetic circuits. Toehold switches, while offering improvements in dynamic range, still rely on single-stranded RNA inputs and can exhibit sequence constraints that limit their versatility. The toehold exchange (THE) riboregulator gate (225) distinguishes itself through its use of a bimolecular strand exchange mechanism. This mechanism, involving the interaction between the gate (225) and an input strand (215), enables precise control over protein translation and offers enhanced modularity for sensing diverse inputs. The gate input toehold domain (210) and the ribosome binding sequence (RBS) sequestering domain (228), which is part of the output strand (201), play crucial roles in this bimolecular reaction. The gate input toehold domain (210) initiates strand exchange with the input strand, while the RBS sequestering domain (228) being released by strand exchange leads to exposure of the ribosome binding sequence (226), directly linking RNA computation to protein production. Furthermore, the inclusion of a self-cleaving ribozyme sequence (209) streamlines riboregulator production by producing the dsRNA gate suitable for strand exchange, enhancing its efficiency and enabling genetic encoding. This combination of features—the bimolecular strand exchange mechanism, the interplay between the toehold domains, RBS sequestering domain, and the RBS, and the co-transcriptional self-cleavage—distinguishes the THE riboregulator from existing technologies and provides a unique approach to controlling gene expression. The tunability of the system, through variations in toehold length, standby domain size, and spacer sequences, further expands its design space and potential applications. The ability to rationally design sequences for specific inputs and protein outputs offers unprecedented control over cellular behavior and expands the possibilities for creating sophisticated genetic control systems. Generally, longer toeholds and shorter distal domains lead to faster kinetics, but longer distal domains can also tune the translation initiation rate.

While one or more embodiments have been shown and described, modifications and substitutions may be made thereto without departing from the spirit and scope of the invention. Accordingly, it is to be understood that the present invention has been described by way of illustrations and not limitation. Embodiments herein can be used independently or can be combined.

All ranges disclosed herein are inclusive of the endpoints, and the endpoints are independently combinable with each other. The ranges are continuous and thus contain every value and subset thereof in the range. Unless otherwise stated or contextually inapplicable, all percentages, when expressing a quantity, are weight percentages. The suffix (s) as used herein is intended to include both the singular and the plural of the term that it modifies, thereby including at least one of that term (e.g., the colorant(s) includes at least one colorants). Option, optional, or optionally means that the subsequently described event or circumstance can or cannot occur, and that the description includes instances where the event occurs and instances where it does not. As used herein, combination is inclusive of blends, mixtures, alloys, reaction products, collection of elements, and the like.

As used herein, a combination thereof refers to a combination comprising at least one of the named constituents, components, compounds, or elements, optionally together with one or more of the same class of constituents, components, compounds, or elements.

All references are incorporated herein by reference.

The use of the terms “a,” “an,” and “the” and similar referents in the context of describing the invention (especially in the context of the following claims) are to be construed to cover both the singular and the plural, unless otherwise indicated herein or clearly contradicted by context. It can further be noted that the terms first, second, primary, secondary, and the like herein do not denote any order, quantity, or importance, but rather are used to distinguish one element from another. It will also be understood that, although the terms first, second, etc. are, in some instances, used herein to describe various elements, these elements should not be limited by these terms. For example, a first current could be termed a second current, and, similarly, a second current could be termed a first current, without departing from the scope of the various described embodiments. The first current and the second current are both currents, but they are not the same condition unless explicitly stated as such.

The modifier about used in connection with a quantity is inclusive of the stated value and has the meaning dictated by the context (e.g., it includes the degree of error associated with measurement of the particular quantity). The conjunction or is used to link objects of a list or alternatives and is not disjunctive; rather the elements can be used separately or can be combined together under appropriate circumstances.

### PARTS LIST

co-transcriptional RNA strand displacement (ctRSD) gate 200
output strand 201
gate prime strand 202
hairpin-forming sequence 203
output branch migration domain 204
output toehold domain 205/genus to 228; complementary to 213
input branch migration domain 206
output wobble domain 207
linker (L) sequence 208
self-cleaving ribozyme sequence 209
gate input toehold domain 210
substrate domain 211
gate prime wobble domain 212
output toehold sequester domain 213/genus to 226; complementary to 205
transcription termination sequence 214
input strand 215
strand exchange product 216
c spacer domain 217
start codon 218
n-terminal linker 219
protein coding sequence (CDS) 220
toehold spacer domain 221
distal (d) ribosome standby domain 222
input toehold domain 223
translation-regulated protein 224
toehold exchange (THE) riboregulator gate (OFF) 225
ribosome binding sequence 226/species of 213, complementary to 228
THE riboregulator gate (ON) 227
ribosome binding sequence (RBS) sequestering domain 228/species of 205, complementary to 226
DNA sequence 300
promoter sequence 301
sequence encoding gate input toehold domain 302
sequence encoding output toehold sequester domain 303
sequence encoding self-cleaving ribozyme sequence 304
sequence encoding start codon 305
sequence encoding c spacer domain 306
sequence encoding protein coding sequence 307
process for regulating protein translation 400
ribosome standby site 401

1. A toehold exchange (THE) riboregulator gate (225) comprising:
a gate input toehold domain (210);
a ribosome binding sequence (226);
a start codon (218);
a c spacer domain (217) positioned between the ribosome binding sequence (226) and the start codon (218);
a protein coding sequence (220); and
a self-cleaving ribozyme sequence (209) that produces a double-stranded RNA (dsRNA) gate suitable for strand exchange and produces the gate input toehold domain (210).

2. The THE riboregulator gate (225) of claim 1, further comprising:
an output strand (201) comprising:
an input branch migration domain (206);
an output branch migration domain (204);
an output toehold domain (205) that sequesters the ribosome binding sequence (226);
a hairpin-forming sequence (203) connected to the output branch migration domain (204);
an output wobble domain (207) connected to the input branch migration domain (206); and
a linker (L) sequence (208) connected to the input branch migration domain (206); and
a gate prime strand (202) hybridized to the output strand (201) comprising:
a gate prime wobble domain (212) connected to a substrate domain (211);
a transcription termination sequence (214); and
the substrate domain (211) connected to the gate input toehold domain (210).

3. The THE riboregulator gate (225) of claim 2, wherein the gate prime wobble domain (212) is connected to the substrate domain (211) through guanine-uracil (G-U) wobble base pairings.

4. The THE riboregulator gate (225) of claim 2, wherein a portion of the input branch migration domain (206) is connected to the output wobble domain (207) through G-U wobble base pairings.

5. The THE riboregulator gate (225) of claim 2, wherein the output strand (201) further comprises a toehold spacer domain (221).

6. The THE riboregulator gate (225) of claim 1, wherein the self-cleaving ribozyme sequence (209) is a hepatitis delta virus (HDV) ribozyme or a ribozyme with an HDV-like fold.

7. The THE riboregulator gate (225) of claim 2, wherein the output toehold domain (205) is complementary to the ribosome binding sequence (226).

8. The THE riboregulator gate (225) of claim 1, wherein the start codon (218) is AUG, GUG, or UUG.

9. The THE riboregulator gate (225) of claim 1, wherein the c spacer domain (217) is 5 to 7 bases in length.

10. The THE riboregulator gate (225) of claim 1, wherein the protein coding sequence (220) encodes a fluorescent protein, an enzyme, or a growth factor.

11. A process for regulating protein translation (400) with a toehold exchange (THE) riboregulator (225) comprising:
providing a THE riboregulator gate (225); and
contacting the THE riboregulator gate (225) with an input strand (215), wherein the input strand (215) comprises an input toehold domain (223) complementary to a gate input toehold domain (210) on the THE riboregulator gate (225).

12. The process of claim 11, wherein the THE riboregulator gate (225) comprises:
a gate input toehold domain (210);
a ribosome binding sequence (226);
a start codon (218);
a c spacer domain (217) positioned between the ribosome binding sequence (226) and the start codon (218);
a protein coding sequence (220); and
a self-cleaving ribozyme sequence (209) that produces a double-stranded RNA (dsRNA) gate suitable for strand exchange and produces the gate input toehold domain (210).

13. The process of claim 12, wherein the self-cleaving ribozyme sequence (209) produces a dsRNA gate suitable for strand exchange and the gate input toehold domain (210).

14. The process of claim 11, wherein the input strand (215) comprises:
a distal (d) ribosome standby domain (222);
an input branch migration domain (206); and
the input toehold domain (223).

15. The process of claim 14, wherein the distal (d) ribosome standby domain (222) is 0 to 20 bases in length.

16. The process of claim 11, further comprising producing a strand exchange product (216) wherein the input strand (215) hybridizes to a gate prime strand (202) and displaces an output strand (201) from the THE riboregulator gate (225), thereby exposing the ribosome binding sequence (226).

17. The process of claim 16, wherein the strand exchange product (216) comprises a THE riboregulator gate (227) in an ON state (227).

18. The process of claim 17, wherein a ribosome standby site (401) is exposed in the THE riboregulator gate (227).

19. The process of claim 11, further comprising initiating translation of the protein coding sequence (220) at the exposed ribosome binding sequence (226) to produce a translation-regulated protein (224).

20. The process of claim 11, wherein the contacting step occurs in a cell-free expression system, in a cell lysate, or in a cell.

21. A process for regulating protein translation with a toehold exchange (THE) riboregulator (225), the process comprising:
providing a THE riboregulator gate (225) comprising:
an output strand (201) comprising the following RNA sequences in sequential order: a linker (L) sequence (208), an output wobble domain (207), an input branch migration domain (206), an output toehold domain (205), an output branch migration domain (204), and a hairpin-forming sequence (203), wherein domains (206) and (204) are partially complementary to an input strand (215) and domain (205) sequesters a ribosome binding sequence (226); and
a gate prime strand (202) hybridized to the output strand (201) comprising the following RNA sequences in sequential order: a self-cleaving ribozyme sequence (209), a gate input toehold domain (210), a substrate domain (211), a gate prime wobble domain (212), a c spacer domain (217), a start codon (218), an n-terminal linker (219), a protein coding sequence (220), a toehold spacer domain (221), and a transcription termination sequence (214), wherein domain (210) is partially complementary to the input strand (215), domain (211) is partially complementary to the input branch migration domain (206) of the output strand (201), and domains (217), (218), (219), and (220) promote translation of the protein coding sequence (220); and
contacting the THE riboregulator gate (225) with an input strand (215) comprising the following RNA sequences in sequential order: an input toehold domain (223), an input branch migration domain (206), and a distal (d) ribosome standby domain (222), wherein domain (223) is complementary to the gate input toehold domain (210) of the gate prime strand (202) and domain (206) is complementary to the substrate domain (211) of the gate prime strand (202);
initiating a strand exchange reaction between the THE riboregulator gate (225) and the input strand (215), wherein the input toehold domain (223) of the input strand (215) hybridizes to the gate input toehold domain (210) of the gate prime strand (202), thereby initiating branch migration between the input branch migration domain (206) of the input strand (215) and the substrate domain (211) of the gate prime strand (202);
displacing the output strand (201) from the gate prime strand (202), thereby forming a strand exchange product (216) comprising a THE riboregulator gate (227) in an ON state (227) with an exposed ribosome binding sequence (226); and
initiating translation of the protein coding sequence (220) at the exposed ribosome binding sequence (226) to produce a translation-regulated protein (224).

22. The process of claim 21, wherein the self-cleaving ribozyme sequence (209) is a hepatitis delta virus (HDV) ribozyme or a ribozyme with an HDV-like fold.

23. The process of claim 21, wherein the gate prime wobble domain (212) is connected to the substrate domain (211) through guanine-uracil (G-U) wobble base pairings.

24. The process of claim 21, wherein a portion of the input branch migration domain (206) is connected to the output wobble domain (207) through G-U wobble base pairings.

25. The process of claim 21, wherein the output toehold domain (205) is complementary to the ribosome binding sequence (226).

26. The process of claim 21, wherein the start codon (218) is AUG, GUG, or UUG.

27. The process of claim 21, wherein the c spacer domain (217) is 5 to 7 bases in length.

28. The process of claim 21, wherein the distal (d) ribosome standby domain (222) is 0 to 20 bases in length.

29. The process of claim 21, wherein the protein coding sequence (220) encodes for a fluorescent protein, an enzyme, or a growth factor.

30. The process of claim 21, wherein the contacting step occurs in a cell-free expression system, in a cell lysate, or in a cell.
